(19) United States
(12) Patent Application Publication
(10) Pub. No.: US 2021/0024885 A1
(43) Pub. Date: Jan. 28, 2021

(54) METHODS AND COMPOSITIONS FOR PRODUCING HEPATOCYTES

(71) Applicant: Ambys Medicines, Inc., South San Francisco, CA (US)

(72) Inventors: Fei Yi, Foster City, CA (US); Raymond D. Hickey, Belmont, CA (US); Michael C. Holmes, Oakland, CA (US)

(21) Appl. No.: 16/938,059

(22) Filed: Jul. 24, 2020

Related U.S. Application Data
(60) Provisional application No. 62/879,142, filed on Jul. 26, 2019.
(60) Provisional application No. 63/000,169, filed on Mar. 26, 2020.

(51) Int. Cl. C12N 5/071; A61K 35/407; A01K 67/027

(52) U.S. Cl. CPC C12N 5/067; A61K 35/407; A01K 67/0275; A01K 2267/025; C12N 2501/11; A01K 2227/105; C12N 2501/12

(57) ABSTRACT

The present disclosure is in the field of methods and compositions for in vivo production of hepatocytes, such as human hepatocytes, as well as uses for the hepatocytes, including e.g., methods involving the administration of hepatocytes to a subject in need thereof, compositions that include such hepatocytes, and the like.

### CROSS-REFERENCE TO RELATED APPLICATIONS

The present application claims the benefit of U.S. Provisional Application No. 62/879,142, filed Jul. 26, 2019 and U.S. Provisional Application No. 63/000,169, filed Mar. 26, 2020, the disclosures of which are hereby incorporated by reference in their entireties.

### TECHNICAL FIELD

The present disclosure is in the field of human hepatocytes, including methods of producing and using these hepatocytes for clinical uses.

### BACKGROUND

Human hepatocytes are widely used by the pharmaceutical industry during preclinical drug development. Indeed, their use is mandated by the FDA as part of drug development. For drug metabolism and other studies, hepatocytes are typically isolated from cadaveric organ donors and shipped to the location where testing will be performed. The condition (viability and state of differentiation) of hepatocytes from cadaveric sources is highly variable and many cell preparations are of marginal quality. The availability of high-quality human hepatocytes is further hampered by the fact that they cannot be significantly expanded in tissue culture (Runge et al. (2000) Biochem. Biophys. Res. Commun. 274:1-3; Cascio et al. (2001) Organs 25:529-538). After plating, the cells survive but do not divide, and lose metabolic functions rapidly. Hepatocytes from readily available mammalian species, such as the mouse, are not suitable for drug testing because they have a different complement of metabolic enzymes and respond differently in induction studies. Immortal human liver cells (hepatomas) or fetal hepatoblasts are also not an adequate replacement for fully differentiated adult cells. Human hepatocytes are also necessary for studies in the field of microbiology. Many human viruses, such as viruses that cause hepatitis, cannot replicate in any other cell type.

Currently, orthotopic liver transplantation remains the only available curative treatment for liver disease. However, this treatment is severely restrained due to the poor availability of high-quality livers. Human hepatocytes cannot be expanded significantly in culture. Hepatocytes derived from stem cells in culture are immature and generally lack full functionality. Therefore, all hepatocytes in use today are derived from human donors, either cadaveric or surgical specimens, which significantly limits hepatocyte availability. Recently, the use of animals for expanding hepatocytes (animals as in vivo bioreactors) has been described, including animal models of hereditary tyrosinemia type 1, which are deficient in FAH, RAG-1 or RAG-2, and IL-2Rγ (Fah−/−, Rag1−/− or Rag2−/−, Il2rg−/− [FRG]). See, e.g., U.S. Pat. No. 8,569,573 (mice); U.S. Pat. No. 9,000,257 (pigs) and U.S. Patent Publication No. 20160249591 (rats). However, currently only about up to 15% of the hepatocytes transplanted into these animal bioreactors are able to survive and engraft (repopulate in the host liver) after transplantation. Furthermore, these Fah-deficient animals require treatment with NTBC (2-nitro-4-trifluoro-methyl-benzoyl)-1,3 cyclohexanedione, also known as nitisinone) to block the tyrosine catabolism pathway to prevent the accumulation of fumarlyacetoacetate. Due to the low yield of well-characterized, functional hepatocytes, to date, no human transplantation using bioreactor expanded human hepatocytes has been reported.

### SUMMARY

Disclosed herein are methods and compositions for enhanced repopulation, engraftment, survival and/or expansion of human hepatocytes transplanted into in vivo bioreactors. Also described herein are isolated populations of these expanded hepatocytes for various uses, including but not limited to use in treatment and/or prevention of liver disease in a human subject.

In one aspect, described herein is a method of producing hepatocytes, the method comprising administering ex vivo manipulated cells that generate hepatocytes (e.g., stem cells, hepatocyte progenitor cells, hepatocyte-like cells, mature or juvenile hepatocytes, etc.) to a live animal such that the hepatocyte-generating cells are expanded in the animal and isolating the expanded hepatocytes from the animal. In certain embodiments, the ex vivo manipulation comprises treating (incubating) isolated hepatocyte-generating cells (e.g., human hepatocytes) with at least one agent that promotes health, growth, regeneration, survival and/or engraftment of hepatocytes and transplanting (e.g., via injection into the spleen or liver) the treated cells into any suitable animal bioreactor (e.g., a genetically modified animal, including but not limited to a FAH-deficient animal such as a pig or rodent). In certain embodiments, the at least one agent that the cells are treated with comprises an antibody, for example at least one c-MET (also referred to as tyrosine-protein kinase Met or hepatocyte growth factor receptor) and/or epidermal growth factor (EGFR) antibody, which may be specific for human cells or may be cross-reactive with two or more species. In certain embodiments, greater than 10%, greater than 15%, 40% or greater, 50% or greater, 70% or greater, 75% or greater, 80% or greater, 85% or greater, and 90% or greater rates of repopulation are achieved in the animal bioreactor. In certain embodiments, the ratio of liver cells derived from transplanted cells to endogenous liver cells is 1:1, 2:1, 3:1 or more, including but not limited to e.g., 1:1 or more, 2:1 or more, 3:1 or more, 4:1 or more, 5:1 or more, 6:1 or more, 7:1 or more, 8:1 or more, 9:1 or more, 10:1 or more, etc. In certain embodiments, the repopulated cells obtained following transplantation of the treated cells into animal bioreactor are healthier (as measured by any suitable qualitative or quantitative assay) than cells derived from transplantation of untreated cells. In certain embodiments, repopulation is achieved within weeks (e.g., 2-16, 2-14, or 2-12 weeks or any time therebetween), months (1 to 12 months or any time therebetween) or years (1 to 5 years or more). In certain embodiments, repopulation rates are achieved weeks (e.g., 2-16, 2-14, or 2-12 weeks or any time therebetween) before rates achieved in which the hepatocyte-generating cells are not treated prior to (and/or after) transplantation with the at least one agent that promotes growth, regeneration, survival and/or engraftment of hepatocytes (e.g., one or more c-MET and/or EGFR antibodies).

In any of the methods of ex vivo manipulation, the hepatocyte-generating cells (e.g., stem cells, hepatocyte progenitor cells, hepatocyte-like cells, mature or juvenile hepatocytes) may be obtained from a commercial source or isolated from live subjects or cadavers. In addition, the hepatocyte-generating cells may be cultured in any media, in some embodiments, the culture media comprises a 1:1 mix completion of HBM Hepatocyte Basal Media and HCM Single™ Quots™ kit (Lonza), 5% FBS, and 10 uM ROCK inhibitor.

In certain aspects, the ex vivo manipulation of hepatocyte-generating cells as described herein comprises adding at least one agent that promotes growth, regeneration, survival and/or engraftment of hepatocytes (e.g., one or more c-MET antibodies) to the cultured hepatocytes and incubating the mixture of hepatocytes and agent for a period of time, including e.g., 1 minute to 2 days (or any time therebetween), 1 to 24 hours (or any time therebetween), 1 to 4 hours (or any time therebetween), etc. In certain embodiments, the cells (e.g., hepatocytes) and agent (e.g., c-MET and/or EGFR antibody) are incubated for 1 hour, optionally with rocking during incubation, which may help maximize exposure of the hepatocytes to the agent.

In any of the methods described herein, the ex vivo manipulated hepatocyte-generating cells are collected and administered to a suitable animal bioreactor for expansion. In certain embodiments, the animal bioreactor comprises a genetically modified animal, for example an animal in which one or more gene targets recombinantly modified, including e.g., where the one or more gene targets are knocked out and/or knocked down. In certain embodiments, multiple genes are modified (e.g., knocked-down and/or activated) in the animal bioreactor. In certain embodiments, the animal bioreactor comprises a genetic modification conferring a deficiency in the production or function of fumarylacetoacetate hydrolase (FAH). Such an animal may be referred to as a fah-deficient animal (including but not limited to, e.g., FRG animals such as rat, mouse or pig). FAH deficiency need not necessarily require genetic modification of a fah locus. For example, in some embodiments, the animal bioreactor comprises a genetically modified animal in which a gene that modifies fah gene expression is modified where the modified gene is not a fah gene, for example a gene upstream of fah that modifies fah expression. The hepatocyte-generating cells may be introduced (transplanted, injected, implanted, etc.) into the bioreactor using any suitable means, optionally via intra-splenic injection, intra-portal vein injection or direct injection into the liver of the animal bioreactor. In certain embodiments, the hepatocyte-generating cells are transplanted into an FRG pig, rat or mouse. In some instances, animals comprising the treated cells maybe cycled on/off NTBC during the expansion period. The transplanted hepatocyte-generating cells treated with the at least one agent (e.g., antibody) can exhibit increased (e.g., 10%, 20%, 30%, 40%, 50%, 60%, 70%, 80%, 90% or more) survival and/or engraftment in the animal bioreactor as compared to animals transplanted with untreated hepatocytes (i.e., hepatocytes not subjected to the ex vivo manipulation as described herein) or increased repopulation rates as compared to animals not subject to transplantation.) The increased engraftment and survival reduces the number of cell cycles/cell divisions needed for the engrafted cells to reach a given repopulation percentage in the animal bioreactor as compared to transplantation of untreated hepatocytes. Without being bound by theory, in some hepatocytes that have undergone fewer cell cycles/cell divisions may be, or may produce progeny that are, healthier, more stable and/or more durable hepatocytes, for example genetically more stable and/or durable. Various measures of cell health, stability and/or durability may be employed to quantitatively or qualitatively show such increased stability and/or durability (e.g., expanded hepatocytes exhibiting longer telomere length, cell proliferation assays, etc.).

In certain embodiments, the ex vivo manipulated hepatocyte-generating cells are expanded in the animal bioreactor for a period of 14-112 days or 28-112 days (2 or 4 to 16 weeks) or any time therebetween, optionally 14-56 days or 28-56 days (2 or 4 to 8 weeks), and harvested (collected) after that time. In certain embodiments, hepatocytes produced in the animal bioreactor are harvested by 8 weeks after transplantation into the animal bioreactor, which hepatocytes have expanded (repopulated) in the animal to more than 50%, more than 60%, more than 70%, or between 80% and 100% of the total hepatocyte population of the animal. In some instances, harvesting by 8 weeks eliminates the need for further NTBC cycling and the long NTBC-off cycle (14 or 21 days) which may dramatically stress animals. Without being bound by theory, in some instances by omitting a long NTBC-off cycle, the health of the animal bioreactor may be improved and, consequently, the health, number, quality, stability and/or durability of the hepatocytes (e.g., human hepatocytes) produced.

In certain embodiments, primary human hepatocytes are administered to (transplanted into) an animal (e.g., rat, mouse, pig, rabbit, etc.) bioreactor and at least 40% repopulation of an animal's liver is achieved (e.g., with NTBC cycling), optionally by 4-16 weeks post-administration. Repopulated human hepatocytes purified from FRG animal livers demonstrate mature hepatic functions in vitro, and robust in vivo potency, including efficient engraftment and expansion in vivo after transplanting into an FRG animal (e.g., mouse, rat, pig, rabbit, etc.). Thus, the FRG animal bioreactors of the described herein generate high-quality primary human hepatocytes suitable for transplantation into patients, thereby providing a therapeutic benefit to a subject with liver disease (and an alternative to liver transplantation).

In another aspect, any of the methods described herein may further comprise ex vivo manipulation of expanded hepatocytes collected from the animal bioreactor, for example culturing (incubating) the expanded hepatocytes with at least one agent that promotes growth, regeneration, survival and/or engraftment of hepatocytes, optionally one or more c-MET antibodies. Further ex vivo manipulation may also comprise introducing one or more genetic modifications to the hepatocytes using known techniques.

In yet another aspect, any of the methods described herein may further comprise repeating the steps one or more times, for example to conduct serial transplantations by introducing the hepatocytes collected from the animal bioreactor and subject to further ex vivo manipulation into the same or different animal bioreactor for further expansion. The steps of the methods may be repeated 1, 2, 3, 4 or more times.

In another aspect, described herein is a method of treating and/or preventing one or more liver diseases or disorders in a subject in need thereof, the method comprising administering the expanded hepatocytes (collected from the animals and with or without further ex vivo manipulation) to subject in need thereof. In certain embodiments, 1×10⁷ to 5×10⁸ cells/kg that represents approximately 1% to 25% of total liver hepatocyte cell mass will be used for transplantation in clinic for a variety of human liver diseases, including but not limited to, chronic liver disease such as cirrhosis, alcoholic hepatitis, hepatic encephalopathy, acute-on-chronic liver failure (ACLF), drug- or poisoning-induced liver failure, and/or one or more inborn metabolic liver diseases.

In another aspect, described herein is an animal bioreactor comprising ex vivo manipulated hepatocytes as described herein. In certain embodiments, the animal bioreactor is a fah-deficient animal (e.g., rat, mouse or pig). In certain embodiments, the animal bioreactor comprising the hepatocytes is subject to treatment with NTBC (e.g., NTBC cycling). NTBC-off cycle provides selection pressure in fah-deficient animals and favors the repopulation (engraftment, survival and/or expansion) of engrafted human hepatocytes. In certain embodiments, more than 50%, more than 60%, more than 70%, or between 80% and 100% of human hepatocyte repopulation rates are achieved in the animal bioreactor by ex vivo manipulated hepatocytes that engraft, survive and/or expand in the animal bioreactor. In certain embodiments, more than 50-70% human hepatocyte repopulation is achieved by 8-16 (or any value therebetween) weeks, for example 70% repopulation by 8-12 weeks (or any value therebetween). See, e.g., FIG. 3B.

In another aspect, described herein is a population of hepatocytes produced using a method as described herein. In certain embodiments, the population of expanded hepatocytes comprises hepatocytes (e.g., human hepatocytes) isolated from an animal bioreactor into which the hepatocytes treated ex vivo as described herein (e.g., with at least one agent as described herein) were administered. The isolated populations comprising expanded hepatocytes as described herein can be used for ex vivo treatment of liver disease in a subject and/or can be further manipulated ex vivo (e.g., via further rounds of the methods described herein) prior to use as an ex vivo treatment. The bioreactor and/or subject comprising the population of hepatocytes may optionally be further treated with one or more agents (e.g., one or more agents as described herein) to further enhance engraftment and/or expansion of the cells in the bioreactor and/or subject. In certain embodiments, the bioreactor and/or subject is optionally administered one or more c-MET antibody (agonists) sequentially (in any order) and/or concurrently with the hepatocytes as treated herein.

In a still further aspect, provided herein is a method of expanding hepatocytes in a human subject, the method comprising administering to the subject human hepatocytes produced in an animal bioreactor as described herein. In certain embodiments, the hepatocytes (including compositions comprising hepatocytes as described herein) are administered through portal vein infusion. In some instances, hepatocytes may be administered via umbilical vein infusion, direct splenic capsule injection, splenic artery infusion, or intraperitoneal injection. Hepatocytes obtained as described herein may or may not be encapsulated prior to administration to the subject. In any of the methods described herein the hepatocytes described herein engraft, survive and/or expand in the subject more efficiently than hepatocytes produced by other methods, in which up to 10% to 15% of hepatocytes transplanted engraft in vivo. In certain embodiments, more than 5%-50% (or any value therebetween), more than 60%, more than 70%, or between 80% and 100% of the hepatocytes transplanted into the patient engraft, survive and/or expand in the patient over time. In some instances, the hepatocytes described herein engraft, survive and/or expand in the subject more efficiently, e.g., at least 1.1-fold more, including e.g., at least 1.2-fold, at least 1.3-fold, at least 1.4-fold, at least 1.5-fold, at least 1.6-fold, at least 1.7-fold, at least 1.8-fold, at least 1.9-fold, at least 2-fold, or at least 2.5-fold more than other methods. In some instances, the hepatocytes described herein engraft, survive and/or expand in the subject more efficiently, e.g., at least 10% more efficiently, including e.g., at least 20%, at least 30%, at least 40%, at least 50%, at least 60%, at least 70%, at least 80%, at least 90%, at least 100%, or at least 150% more efficiently than other methods. In any of the methods described herein, following administration to the subject, over any time period (including but not limited to 2-16 weeks, 2-14 weeks, 2-12 weeks, 1-12 months or over year), the hepatocytes as described herein comprise at least 5%, at least 10% or more of the total number of cells in the subject's liver.

In yet another aspect, provided herein is a method of treating and/or preventing liver disease in a human subject in need thereof, the method comprising administering to the subject a population of expanded human hepatocytes as described. Thus, the methods described herein can be used for hepatocyte cell therapy in clinic by providing healthy hepatocytes and as a stand-alone therapy, which, due to the enhanced engraftment and/or repopulation profile results in more efficient disease treatment and/or prevention than current methods using fresh or cryopreserved hepatocytes. Administration may be by any suitable means, including but not limited to intravenous (e.g., portal vein), intraperitoneal, into the omental bursa, transplantation and/or implantation into one or more organs or tissues (e.g., liver, spleen, lymph nodes, etc.).

In any of the methods described herein involving a subject, the methods may further comprise administering one or more agents (e.g., antibodies, small molecules, nucleic acids (DNA and/or RNA), etc.) that promote growth, regeneration, survival and/or engraftment of hepatocytes in the subject. In certain embodiments, at least one agent comprises a c-MET antibody, optionally one that is human-specific. The one or more agents may be administered one, two or more times and may be administered with and/or at different times than the hepatocytes.

Furthermore, any of the ex vivo methods involving administration of expanded hepatocytes to a subject may further comprise repeating one or more steps of the methods, including for example repeated administration (2, 3, 4, 5, 6, 7 or more administrations) of the expanded hepatocytes as described herein at any time interval(s).

Disease and disorders that can be treated by the methods and compositions described herein include but are not limited to Crigler-Najjar syndrome type 1; familial hypercholesterolemia; Factor VII deficiency; Factor VIII deficiency (Hemophilia A); Phenylketonuria (PKU); Glycogen storage disease type I; infantile Refsum's disease; Progressive familial intrahepatic cholestasis type 2; hereditary tyrosinemia type 1; and various urea cycle defects; acute liver failure, including juvenile and adult patients with acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including cirrhosis; acute-on-chronic liver disease caused by one of the following acute events: alcohol consumption, drug ingestion, and/or hepatitis B flare ups. Liver diseases that may be treated and/or prevented using the methods and compositions described herein thus include both liver diseases in which the transplanted (manipulated) cells are not, or are not expected to be, injured after transplantation into livers in which the endogenous hepatocytes are injured/diseased (also referred to as “endogenous liver disease”) as well as liver diseases in which the transplanted (manipulated) cells and endogenous hepatocytes may both be subject to, or affected by, injury/disease, for example by extrinsic factors (also referred to as “exogenous liver disease”).

Described herein are methods of producing hepatocytes, the methods comprising: manipulating hepatocyte-generating cells (e.g., primary human hepatocytes) by contacting the hepatocyte-generating cells ex vivo with at least one agent that promotes growth, regeneration, survival and/or engraftment (e.g., an agonist that specifically binds to a growth factor receptor such as c-MET and/or EGFR, optionally a small molecule or an antibody); transplanting the ex vivo manipulated cells into an in vivo bioreactor under conditions suitable for engraftment; and maintaining the in vivo bioreactor under conditions suitable to expand the engrafted cells into an expanded hepatocyte population in the bioreactor, optionally increasing engraftment and/or repopulation efficiency of the expanded cells by at least 10% as compared to a corresponding method lacking the ex vivo manipulation. In certain embodiments, the hepatocyte-generating cells and the at least one agent are contained within a vessel and the incubating comprises agitating the vessel, optionally wherein the agitating comprises rocking. Any of the methods described herein may further comprise separating the at least one agent from the ex vivo manipulated cells prior to the transplanting, for example by removing the at least one agent and/or isolating the ex vivo manipulated cells, optionally via centrifugation and/or aspiration. Any of the methods described herein may further comprise isolating the expanded hepatocytes (e.g., from the bioreactor). In any of the methods described herein, the engrafted cells are expanded for a period of anywhere between about 2 to 16 weeks. In any of the methods described herein, the expanded hepatocytes comprise at least 50% of the total hepatocyte population of the in vivo bioreactor. The in vivo bioreactor may be a mammal and optionally may have an endogenous liver injury and/or be immunosuppressed, optionally a mouse, rat or pig bioreactor comprising a FAH deficiency, an IL-2Rγ deficiency, a RAG1 deficiency, a RAG2 deficiency, or any combination thereof (e.g., a rodent or pig comprising a FAH, RAG1 and/or RAG2, and IL-2Rγ deficiency (FRG)).

In yet another aspect, described herein are methods of treating a subject for a liver disease, the method comprising: administering ex vivo manipulated cells that generate hepatocytes to the subject in an amount effective to engraft and expand in vivo thereby treating the liver disease in a subject. The ex vivo manipulated cells are produced by any of the methods or systems described herein, for example by incubating hepatocyte-generating cells with at least one agent that promotes growth, regeneration, survival and/or engraftment and expanding the ex vivo manipulated cells in an in vivo bioreactor prior to administration to the subject. The liver disease(s) that may be treated include, but are not necessarily limited to, inherited disorders, liver failure, liver disease caused by an enzyme deficiency, including but not limited to: cirrhosis; acute-on-chronic liver failure (ACLF); drug- or poisoning-induced liver failure; an inborn metabolic liver disease; Crigler-Najjar syndrome type 1; familial hypercholesterolemia; Factor VII deficiency; Factor VIII deficiency (Hemophilia A); Phenylketonuria (PKU); Glycogen storage disease type I; infantile Refsum's disease; Progressive familial intrahepatic cholestasis type 2; hereditary tyrosinemia type 1; a urea cycle defect; acute liver failure; acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including alcoholic hepatitis, hepatic encephalopathy, cirrhosis; and/or acute-on-chronic liver disease caused alcohol consumption, drug ingestion, and/or hepatitis B flare ups. Any of the methods described herein can result in prolonged survival of the subject, optionally as compared to survival of a comparable subject not administered the ex vivo manipulated cells, optionally administered cells that have not been ex vivo manipulated as described herein.

Also provided are uses of cells (e.g., populations of cells and compositions comprising these cells) as described herein, including cells (and compositions containing these cells) produced by any of the methods as described herein for the treatment of liver disease, including in the preparation of medicament for treatment of one or more liver diseases, including but not limited to inherited disorders, liver failure, liver disease caused by an enzyme deficiency, such as: cirrhosis; acute-on-chronic liver failure (ACLF); drug- or poisoning-induced liver failure; an inborn metabolic liver disease; Crigler-Najjar syndrome type 1; familial hypercholesterolemia; Factor VII deficiency; Factor VIII deficiency (Hemophilia A); Phenylketonuria (PKU); Glycogen storage disease type I; infantile Refsum's disease; Progressive familial intrahepatic cholestasis type 2; hereditary tyrosinemia type 1; a urea cycle defect; acute liver failure; acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including alcoholic hepatitis, hepatic encephalopathy, cirrhosis; and/or acute-on-chronic liver disease caused alcohol consumption, drug ingestion, and/or hepatitis B flare ups.

In yet another aspect, provided herein is a kit comprising hepatocyte-generating cells (e.g., human hepatocytes) and/or at least one agent that promotes growth, regeneration, survival and/or engraftment of hepatocytes, optionally comprising instructions for performing the methods of the present disclosure and producing the compositions described herein. In certain embodiments, the hepatocytes are expanded hepatocytes as described herein.

These and other aspects will be readily apparent to the skilled artisan in light of disclosure as a whole.

### BRIEF DESCRIPTION OF THE DRAWINGS

FIG. 1 is a schematic, adapted from Lee, et al. (2015) Immunotargets Ther. 4:35-44, depicting the HGF/c-MET signaling pathway. Abbreviations used in the Figure are as follows: “AKT” refers to Ak strain transforming; protein kinase B; “c-MET” refers to mesenchymal-epithelial transition factor; hepatocyte growth factor receptor; “GRB2” refers to growth factor receptor-bound protein 2; “GAB1” refers to GRB2-associated binding protein 1; “HGF” refers to hepatocyte growth factor; “mTOR” refers to mammalian target of rapamycin; “MAPK” refers to mitogen-activated protein kinase; “PI3K” refers to phosphatidylinositol-4,5-bisphosphate 3-kinase; “STAT3” refers to signal transducer and activator of transcription 3.

FIG. 2A through FIG. 2F depict ex vivo manipulation of primary human hepatocytes (PHH) with c-MET agonist antibody leading to increased levels of engraftment and expansion in FRG mice. FIG. 2A shows the percent of FAH positive (FAH+) human hepatocytes in FRG mouse livers 1-week after transplantation of primary human hepatocytes with (open circles) or without (shaded circles) c-MET antibody manipulation. Each data point represents a single animal. FIG. 2B shows examples of FAH immunohistochemistry imaging of FRG mouse livers of the indicated conditions 1-week post-transplantation. Doublets of FAH+ human hepatocytes were only observed, at 2 weeks, in liver transplanted with c-MET antibody treated hepatocytes. The image on the left shows results following transplantation of cells not treated with C-MET antibody (“No Ab Ctrl”) and the image on the right shows results following transplantation of cells treated with c-MET antibody (“c-MET Ab”). FIG. 2C shows the percent of FAH positive human hepatocytes (top graph) and human albumin levels measured in blood (bottom graph) in FRG mice 2-weeks post-transplantation of cells treated with (open circles) or without (shaded circles) a c-MET antibody. Each data point represents a single animal. FIG. 2D shows examples of FAH immunohistochemistry of FRG mouse livers of the indicated conditions at 2-weeks post-transplantation. FIG. 2E shows the percent of FAH positive human hepatocytes (top graph) and human albumin levels measured in blood (bottom graph) in FRG mice at 4-weeks post-transplantation. Each data point represents a single animal (shaded circles depict control animals that received cells not treated with c-MET antibody and open squares depict animals that received cells treated with c-MET antibody). FIG. 2F shows examples of FAH immunohistochemistry of FRG mouse livers administered cells of the indicated conditions 4-weeks post-transplantation. The top image shows results following transplantation of cells not treated with C-MET antibody (“No Ab Ctrl”) and the bottom image shows results following transplantation of cells treated with c-MET antibody (“c-MET Ab”).

FIG. 3 depicts ex vivo manipulation of primary human hepatocytes with c-MET agonist antibodies which lead to increased levels of repopulation in FRG mice. Current methods of hepatocyte production in FRG mice involve levels of cell engraftment following transplantation corresponding to less than about 1% liver repopulation (e.g., about 20-50 μg/mL human albumin (hALB) at 4 weeks post-transplantation and about 1-5% liver repopulation (e.g., 200-500 μg/mL hALB) after about 8 weeks. FRG mice have been observed to reach about 20-95% liver repopulation (e.g., 2000-5000+μg/mL hALB); however, this range is generally not obtained until after about 12+ weeks. FIG. 3 shows FAH+ human hepatocyte repopulation at 8 weeks post-transplantation in FRG mouse livers, through exemplary FAH immunohistochemistry of liver sections taken from mice administered cells treated as indicated. For comparison, the left panel (“no Ab control”) shows FRG mouse liver transplanted with human hepatocytes treated using current methods prior to transplantation (i.e., without ex vivo manipulation as described herein). The middle panel (“c-MET Ab_1”) and right (“c-MET Ab_2”) panel show results of FRG mouse livers transplanted with human hepatocytes manipulated ex vivo prior to transplantation as described herein. Specifically, hepatocytes were treated with one of two different c-MET agonist antibodies as indicated (i.e., “Ab_1” or “Ab_2”). Also shown below each panel is the percentage of FAH+ human hepatocytes repopulated in mouse liver (assessed by IHC) and human albumin levels measured in blood (by ELISA). As shown, ex vivo manipulation of the hepatocytes as described herein resulted in ˜90% repopulation with transplanted hepatocytes as compared to less than ˜17% repopulation in animals that received hepatocytes that were not subjected to the ex vivo manipulation described herein. Also shown is that Human albumin levels were significantly increased in animals that received the ex vivo manipulated hepatocytes as compared to the control animals that received hepatocytes not treated with a c-MET antibody.

FIG. 4 shows graphs depicting that ex vivo manipulation of primary human hepatocytes with EGFR agonist antibody leads to increased levels of engraftment and expansion in FRG mice. Human albumin levels, measured from blood in FRG mice 4-weeks (left graph) and 8-weeks (right graph) post-transplantation of human hepatocytes that were or were not (as indicated) subjected to ex vivo manipulation with EGFR agonist antibody as described herein. Each graph shows results following transplantation of cells not treated with EGFR antibody (“No Ab Ctrl”) and results following transplantation of cells treated with EGFR antibody (“EGFR Ab”). Each data point represents a single animal.

FIG. 5 shows graphs depicting that ex vivo manipulation of primary human hepatocytes with both c-MET and EGFR agonist antibodies leads to increased levels of engraftment and expansion in FRG mice. The percent of FAH positive human hepatocytes (left graph) and human albumin levels measured in blood (right graph), in FRG mice transplanted with human hepatocytes not treated with antibodies (shaded circles labeled “No Ab Ctrl”); human hepatocytes ex vivo manipulated with c-MET antibody alone (open circles labeled “c-MET Ab”); or c-MET and EGFR antibodies (open circles with dashed borders labeled “c-MET+EGFR Ab”), 2-weeks post-transplantation are provided. Each data point represents a single animal. t-test between groups: * p<0.05; ** p<0.01.

FIG. 6 is a schematic depicting exemplary ex vivo manipulation of hepatocytes as described herein in a rodent (e.g., mouse or rat) bioreactor. As shown, human hepatocytes may be manipulated ex vivo before and/or after transplantation into a rodent bioreactor. Following expansion in the bioreactor, in some instances, expanded hepatocytes may be administered to a subject, including e.g., adult and/or pediatric subjects. As shown, hepatocytes may or may not be serially transplanted into an animal bioreactor for further expansion (with or without additional rounds of ex vivo manipulation). Also pictured is ex vivo manipulation that may or may not be performed prior to administration of expanded hepatocytes to a subject in need thereof, such as the human subjects as shown, to treat the subject for a condition, such as e.g., liver disease.

### DETAILED DESCRIPTION

Orthotopic liver transplantation remains the only curative treatment for liver disease. Hepatocyte transplantation is a potential alternative therapy for acute and chronic liver diseases; however, obtaining functional hepatocytes is difficult due to the poor availability of high-quality livers and low yield obtained from livers.

Disclosed herein are methods of producing, including expanding, hepatocytes for various purposes. In some instances, the instant methods provide for the production and/or expansion of human hepatocytes suitable for transplantation into a subject in need thereof, including human hepatocytes suitable for orthotopic liver transplantation. Hepatocytes, including human hepatocytes, produced according to the methods described herein can be purified, cryopreserved, and/or extensively characterized prior to infusion. Among other uses, hepatocytes produced according to the methods described herein may provide on-demand therapy for patients with one or more severe liver diseases.

Also provided herein are compositions comprising hepatocytes produced and/or expanded according to the methods as described herein. The compositions and methods described herein, in some embodiments, contain and produce human hepatocytes suitable for transplantation into patients with one or more liver disorders. In some instances, a composition administered to a subject, as described herein, will include hepatocyte-generating cells that have been ex vivo manipulated to enhance engraftment and/or expansion of such cells within the subject. In some instances, a composition administered to a subject, as described herein, will include a population of hepatocytes that have been expanded in an in vivo bioreactor following ex vivo manipulation to enhance engraftment and/or expansion of such cells within the bioreactor. As such, ex vivo manipulation to enhance engraftment and/or expansion may be utilized at various points in the processes described herein, including e.g., before expansion in a bioreactor, before transplantation into a subject, both before expansion in a bioreactor and before transplantation into a subject, and the like.

Methods described herein, in some instances, involve expansion of exogenous hepatocytes in an in vivo bioreactor, including wherein the exogenous hepatocytes repopulate the host liver achieving repopulation rates of greater than 40%, 50% or more, 55% or more, 60% or more, 65% or more, 70% or more, 75% or more, 80% or more, 85% or more, 90% or more, or 95% or more. In some instances, a repopulated liver (e.g., an FGR rodent liver) may comprise greater than 80% repopulated hepatocytes (including e.g., 85% or greater, 90% or greater, 95% or greater), whereas 100% repopulation would represent a liver having a hepatocyte population completely derived from exogenous, transplanted hepatocyte-generating cells (i.e., devoid of host-derived hepatocytes). Further, in some embodiments, the methods described herein produce large quantities of these human hepatocytes more quickly than current methods, including achieving repopulation rates of 40%, 60%, 80% or more by 8 weeks, e.g., as compared to current methods in which less than 20% repopulation rates are achieved 8 weeks post-engraftment. This disclosure thus provides a source of well characterized, mature, functional human hepatocytes for treatment of patients with liver disease(s).

In one aspect, disclosed herein are compositions and methods for the production of hepatocytes, particularly the expansion of human hepatocytes following transplantation of hepatocyte-generating cells into animal bioreactors.

The present disclosure provides significant and unexpected advantages as compared to currently used protocols and compositions, including, but not limited to: (1) significantly enhanced survival, engraftment and/or repopulation of hepatocytes in animal bioreactors (e.g., FRG animals); (2) reducing the time required in the animal to achieve optimal (70-90%) repopulation (thereby reducing costs associated with animal facilities and/or reagents administered to the animals); (3) reducing the number of hepatocytes needed for transplantation (reducing cost associated with obtaining hepatocytes); (4) reducing the need for NTBC cycling in the animal bioreactor (thereby improving the health of the animal bioreactor and the quality of the hepatocytes obtained); (5) retaining proliferation potential of hepatocytes expanded in the bioreactor by reducing number of cell division during clonal expansion; (6) reducing the amount of cell purification required from the animal bioreactor (e.g., by increasing the percentage of desired cells present in the bioreactor at harvest); (7) increasing the quality (e.g., as determined by albumin production levels of the cells, assaying cell viability and/or platability of the purified cells) of hepatocytes purified from the animal bioreactor and/or (8) providing a potential as a stand-alone antibody therapy for liver diseases by directly treating patients with recurring administrations of the antibody to promote liver regeneration; (9) providing a potential to combine the ex vivo manipulation and in vivo administration of the agent to further improve human hepatocytes repopulation in bioreactor and in clinic; and/or (10) an improved a cell therapy for liver diseases characterized by increased repopulation in subjects receiving ex vivo manipulated hepatocyte-generating cells, resulting in enhanced therapeutic outcomes.

Thus, the methods and composition described herein as ex vivo manipulation can improve human hepatocyte repopulation in animal (e.g., rodent or pig) bioreactors. In addition, following isolation from the bioreactors, the hepatocytes produced by the methods described herein exhibit increased functionality and repopulation efficiency, providing concordant improvements when administered to human subjects for the treatment and/or prevention of liver disease.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; and METHODS IN MOLECULAR BIOLOGY, Vol. 119, “Chromatin Protocols” (P. B. Becker, ed.) Humana Press, Totowa, 1999.

### Definitions

The terms “bioreactor, “animal bioreactor”, and “in vivo bioreactor”, as used herein, generally refer to a living non-human animal into which exogenous cells, such as hepatocyte-generating cells, are introduced for engraftment and expansion, thereby generating an expanded population of the cells and/or their progeny, such as an expanded population of hepatocytes, generated from the introduced cells. Introduction of exogenous cells, such as hepatocyte-generating cells, into the bioreactor will generally involve xenotransplantation and, as such, the transplanted exogenous cells may, in some instances, be referred to as a xenograft, e.g., human-to-rodent xenograft, human-to-mouse xenograft, human-to-rat xenograft, human-to-porcine xenograft, mouse-to-rat xenograft, rat-to-mouse xenograft, rodent-to-porcine xenograft, etc. In some instances, allotransplantation into a bioreactor may be performed, e.g., rodent-to-rodent, porcine-to-porcine, etc., allotransplantations. Discussed in more detail herein, a bioreactor may be configured, e.g., genetically and/or pharmacologically, to confer a selective advantage to introduced exogenous cells, such as introduced exogenous hepatocyte-generating cells, in order to promote engraftment and/or expansion thereof. Bioreactors may, in some instances, be configured to prevent rejection of introduced exogenous cells, including but not limited to e.g., through genetic and/or pharmacological immune suppression as described in more detail herein.

The term “ex vivo” is used to refer to handling, experimentation and/or measurements done in or on samples (e.g., tissue or cells, etc.) obtained from an organism, which handling, experimentation and/or measurements are done in an environment external to the organism. Thus, the term “ex vivo manipulation” as applied to cells refers to any handling of the cells (e.g., hepatocytes) outside of an organism, including but not limited to culturing the cells, making one or more genetic modifications to the cells and/or exposing the cells to one or more agents that promote growth, regeneration, survival and/or engraftment when the cells are placed back into an organism (e.g., animal bioreactor or human subject). Accordingly, ex vivo manipulation may be used herein to refer to treatment of cells that is performed outside of an animal, e.g., after such cells are obtained from an animal or organ (e.g., liver) thereof and before such cells are transplanted into an animal, such as an animal bioreactor or subject in need thereof. In contrast to “ex vivo”, the term “in vivo”, as used herein, may refer to cells that are within an animal, or an organ thereof, such as e.g., cells (e.g., hepatocytes) that are within a subject, or the liver thereof, due to generation of the cells within the subject and/or transplantation of the cells into the subject.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acid.

The term “antibody” refers to a protein (or protein complex) that includes one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as the myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one “light” (about 25 kDa) and one “heavy” (about 50-70 kDa) chain. The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains.

As used herein, the term “antibodies” includes intact immunoglobulins as well as a number of well-characterized fragments. For instance, Fabs, Fvs, and single-chain Fvs (scFvs) that bind to a target protein (or an epitope within a protein or fusion protein) would also be specific binding agents for that protein (or epitope). These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody, a genetically engineered molecule containing the variable region of the light chain, the variable region of the heavy chain, linked by a suitable polypeptide linker as a genetically fused single chain molecule. Methods of making these fragments are routine (see, for example, Harlow and Lane, Using Antibodies: A Laboratory Manual, CSHL, New York, 1999).

Antibodies can be monoclonal or polyclonal. Merely by way of example, monoclonal antibodies can be prepared from murine hybridomas according to the classical method of Kohler and Milstein (Nature 256:495-97, 1975) or derivative methods thereof. Detailed procedures for monoclonal antibody production are described in Harlow and Lane, Using Antibodies: A Laboratory Manual, CSHL, New York, 1999. Antibodies can also be “heavy chain only” antibodies or derivatives thereof, such as but not limited to e.g., camelid heavy chain only antibodies, nanobodies, and the like. The term “nanobody”, as used herein, refers to the smallest antigen binding fragment or single variable domain (V_{HH}), e.g., as derived from naturally occurring heavy chain antibodies which may contain a V_{HH} and constant domains (e.g., C_{H}2 and C_{H}3). Nanobodies may be derived from heavy chain only antibodies, seen in camelids (see e.g., Hamers-Casterman et al., 1993; Desmyter et al., 1996), where immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). Heavy-chain antibodies may also be obtained, or derived from, cartilaginous fish antibodies, such as e.g., IgNAR antibodies and fragments thereof, such as V_{NAR} fragments. A single-domain antibody (sdAb) may be referred to as a nanobody or a V_{HH} antibody and such antibodies may be derived through various means, including e.g., from heavy-chain antibodies, from engineering of multi-chain antibodies (such as e.g., mouse, rabbit, or human antibodies), from screening VH domain libraries, and the like.

The terms “sample” and “biological sample” refer to material obtained from cells, tissue or bodily fluid of a subject, such as peripheral blood, serum, plasma, cerebrospinal fluid, bone marrow, urine, saliva, tissue biopsy, surgical specimen, and autopsy material. A sample may also refer to a tissue sample, such as, but not limited to, a liver tissue sample. Tissue samples may be kept and/or utilized in a variety of states including e.g., as intact tissue, as tissue sections, as homogenized tissue, as dissociated and/or purified cells obtained from tissue, etc., which may be prepared according to a variety of techniques including but not limited to e.g., surgical resection, sectioning, homogenization, dissociation, purification, and the like.

As used herein, the term “collecting”, for example as it refers to expanded human hepatocytes, refers to the process of removing the expanded hepatocytes from an animal (e.g., mouse, rat, or pig bioreactor) that has been injected or transplanted with isolated human hepatocytes, or other hepatocyte-generating cells, as described herein. In some instances, a non-human animal that receives a transplantation of cells, e.g., ex vivo manipulated cells, may also be referred to as a recipient animal. In some instances, a human subject that receives a transplantation, e.g., of expanded hepatocytes, may be referred to as a treated subject, a recipient, or the like. Collecting optionally includes separating hepatocytes from other cell types, including but not limited to e.g., non-hepatic cells types (e.g., blood cells, extra-hepatic immune cells, vascular cells, etc.), non-hepatocyte hepatic cells (e.g., hepatic stellate cells, Kupffer cells, and liver sinusoidal endothelial cells).

As used herein, “cryopreserved” refers to a cell (such as a hepatocyte) or tissue that has been preserved or maintained by cooling to low sub-zero temperatures, such as 77 K or −196° C. (the boiling point of liquid nitrogen). At these low temperatures, any biological activity, including the biochemical reactions that would lead to cell death, is effectively stopped. Useful methods of cryopreservation and thawing cryopreserved cells, as well as processes and reagents related thereto, include but are not limited to e.g., those described in U.S. Pat. Nos. 10,370,638; 10,159,244; 9,078,430; 7,604,929; 6,136,525; and 5,795,711, the disclosures of which are incorporated herein by reference in their entirety. In contrast, the term “fresh”, as used herein with reference to cells, may refer to cells that have not been cryopreserved and, e.g., may have been directly obtained and/or used (e.g., transplanted, cultured, etc.) following collection from a subject or organ thereof.

The term “survival” is used to refer to the cells that continue to live after transplantation into the animal, typically including cells that engraft following administration of the cell (e.g., injection) into the animal. Cell survival may be assessed using a variety of methods, including direct assessments (such as e.g., qualitative or quantitative measurements of cell viability in a sample containing or expected to contain the cells of interest) and indirect assessments (such as e.g., qualitative or quantitative measurements of one or more functional consequences of the presence of the viable cell in an animal or human subject). Useful direct and indirect readouts of cell (e.g., hepatocyte) survival may include but are not limited to, cell counting (e.g., via hemocytometer, immunohistochemistry, flow cytometry, etc.), measuring a secreted factor or biomarker (e.g., via protein (e.g., albumin) ELISA, Western blot, etc.), assessing health of a recipient (for example by measuring vitals, function tests (e.g., liver function tests), etc.), and the like. The term “survival” is also used herein to refer to the length of time a subject, e.g., a subject with a liver disease or an animal model thereof, continues to live after some treatment, intervention, and/or challenge, such as e.g., administration or transplantation of cells (e.g., hepatocytes) to the subject, administration of a disease (e.g., liver disease) causing agent to the subject, withdrawal of an agent that inhibits, delays, avoids or prevents the development of disease (e.g., liver disease). Survival, as it refers to subject, may also be expressed in terms of the portion (e.g., percentage) of a population (e.g., a control or treatment group) that lives for a given period of time after some treatment, intervention, and/or challenge. One skilled in the biomedical arts will readily discern wherein survival pertains herein to cells or subjects.

The term “engraft” refers to the implantation of cells or tissues in an animal. As used herein, engraftment of human hepatocytes in a recipient animal refers to the process of human hepatocytes becoming implanted (e.g., in the liver) in the recipient animal following administration (e.g., injection). Under certain conditions engrafted human hepatocytes are capable of expansion in the recipient animal. As used herein, the term “expanding” human hepatocytes refers to the process of allowing cell division to occur such that the number of human hepatocytes increases. The term “in vivo expansion” refers to the process of allowing cell division of exogenous cells to occur within a living host (e.g., a non-human animal bioreactor, such as by way of example, a rodent (e.g., mouse or rat) bioreactor, a pig bioreactor, a rat bioreactor or the like, such that the number of exogenous cells increases within the living host. For example, human hepatocytes transplanted into a non-human animal bioreactor may undergo in vivo expansion within the bioreactor such that the number of human hepatocytes within the bioreactor increases.

The term “repopulation” refers generally to cells that engraft, survive and expand following introduction into an animal (e.g., bioreactor and/or subject). Thus, the term encompasses engrafted cells that expand and proliferate in the animal, including human hepatocytes that expand and proliferate in the liver of the animal. Repopulation, and enhancement thereof, may be described in terms of efficiency, including e.g., where cells with enhanced repopulation kinetics may be said to have increased repopulation efficiency which may result from an improvement or improvements in engraftment, cell survival, proliferation, or some combination thereof. Repopulation may be referred to as a ratio, for example a percentage of total liver cells, or a subpopulation thereof (e.g., percentage of total hepatocytes), following administration to the animal and/or as a percentage of the total liver volume. With regards to transplanted hepatocytes specifically, levels of repopulation will, unless denoted otherwise, generally refer to the ratio of hepatocytes present in the host liver derived from the transplant (i.e., the surviving and engrafted transplanted cells plus any progeny thereof) to host liver cells, or a subpopulation thereof (e.g., host hepatocytes). This ratio may be expressed as a percentage, e.g., where 50% repopulation would represent a host liver that is comprised of cells that are half transplant-derived and half host-derived whereas 100% repopulation would represent a host liver having only transplant-derived hepatocytes. Alternatively, this ratio may be referred to as proportion of cells derived from transplanted cells to cells derived from endogenous cells (e.g., 1:1, 2:1, 3:1, etc.). Repopulation is typically determined after a period of time sufficient for the cells to engraft and expand in the animal, including but not limited to 2-16 weeks, 2-14 weeks, or 2-12 weeks (or any time therebetween), 1-12 months (or any time therebetween), or a year or more. In some instances, repopulation is measured at 2-6 weeks, 6-12 weeks, 4-8 weeks, 6-10 weeks, 8-12 weeks, 10-14 weeks, 12-16 weeks, 14-18 weeks, 2-4 weeks, 2-6 weeks, 6-8 weeks, 8-10 weeks, 10-12 weeks, 12-14 weeks, 14-16 weeks, 16-18 weeks, 18-20 weeks, 1-2 weeks, 2-3 weeks, 3-4 weeks, 4-5 weeks, 5-6 weeks, 6-7 weeks, 7-8 weeks, 8-9 weeks, 9-10 weeks, 10-11 weeks, 11-12 weeks, 12-13 weeks, 13-14 weeks, 14-15 weeks, 15-16 weeks, 17-18 weeks, 18-19 weeks, 19-20 weeks, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks post-transplantation. In some instances, repopulation, where for example repopulation in a first group (e.g., a group receiving ex vivo manipulated cells) is compared to a second group (e.g., a group receiving cells not manipulated ex vivo), may be expressed as having reached a particular level by a certain timepoint, including e.g., at least 20%, at least 30%, at least 40%, at least 50%, at least 60%, at least 70%, at least 75%, at least 80%, at least 85%, or at least 90% or more by 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks or more post-transplantation.

Repopulation may be assessed using a variety of methods, including direct and indirect assessments. Useful direct assessments may include, e.g., qualitative or quantitative measurements of the presence of exogenously-derived cells in a sample containing or expected to contain such cells. The term “exogenously-derived” as used herein with reference to cells, and specifically hepatocytes in some instances, collectively refers to the cells transplanted into a host organism as well as any progeny of such transplanted cells. Accordingly, exogenously-derived cells may refer to the initial hepatocyte-generating cells transplanted into a host as well as any hepatocytes produced during the expansion of such cells. Exogenously-derived cells may be identified by a variety of methods, including but not limited to e.g., staining for or labeling a gene or gene product specifically present or expressed in the exogenously-derived cells (such as e.g., the fah gene, FAH mRNA, or FAH protein expressed in cells transplanted into a FAH deficient (e.g., fah^{-/-}) host). For example, in some embodiments, the level of repopulation may be determined by computing the ratio of the amount of transplant-derived hepatocytes (e.g., as determined by human FAH+ immunohistochemistry (IHC)) in the liver or a sample thereof to the total amount of cells or hepatocytes (e.g., as determined by counter staining, nuclei and/or cytoplasm labeling/counting, or the like) in the liver or sample thereof, optionally expressed as a percent or ratio.

Useful indirect assessments of repopulation may include, e.g., qualitative or quantitative measurements of one or more functional consequences of the presence of the repopulating cell type in an animal or human subject, including but not limited to cell counting (e.g., via hemocytometer, IHC, flow cytometry, etc.), measuring a secreted factor or biomarker (e.g., via protein (e.g., albumin) ELISA, Western blot, etc.). assessing health of the transplanted cells (e.g., via cellular proliferation assays such as enzymatic assays such as MTT, imaging methods, or real-time plate-based assays that are capable of quantitatively measuring cell health), and/or assessing health of the animal bioreactor and/or a recipient (e.g., measuring vitals, function tests (e.g., liver function tests), etc.), and the like.

Direct and indirect readouts of repopulation (e.g., hepatocyte repopulation) may make use of various assays, or combinations thereof, including but not limited to e.g., cell counting (e.g., via hemocytometer, IHC, flow cytometry, etc.), cell staining (e.g., utilizing colorimetric or fluorescent dyes, including e.g., nuclear dyes, cytoplasmic dyes, histological stains, etc.), cell labeling (e.g., through the use of detectable specific binding agents, such as e.g., detectable antibodies and the like), measuring one or more secreted factors or biomarkers (e.g., via protein (e.g., albumin) ELISA, Western blot, etc.), detecting and/or quantifying nucleic acids (e.g., DNA or RNA, e.g., via in situ hybridization, qPCR, sequencing, etc.), assessing the health of a recipient (e.g., measuring vitals, function tests (e.g., liver function tests), etc.), survival assays, and the like.

The term “hepatocyte” refers to a type of cell that generally makes up 70-80% of the cytoplasmic mass of the liver. Hepatocytes are involved in protein synthesis, protein storage and transformation of carbohydrates, synthesis of cholesterol, bile salts and phospholipids, and detoxification, modification and excretion of exogenous and endogenous substances. The hepatocyte also initiates the formation and secretion of bile. Hepatocytes manufacture serum albumin, fibrinogen and the prothrombin group of clotting factors and are the main site for the synthesis of lipoproteins, ceruloplasmin, transferrin, complement and glycoproteins. In addition, hepatocytes have the ability to metabolize, detoxify, and inactivate exogenous compounds such as drugs and insecticides, and endogenous compounds such as steroids.

The terms “subject” and “subjects” are used interchangeably and refer to mammals such as human subjects and non-human primates, as well as experimental animals such as rabbits, dogs, cats, rats, mice, pigs, and other animals. Accordingly, the term “subject” or “subjects” as used herein means any mammalian subject or subject to which the cells described herein can be administered. Subjects of the present disclosure include those having a liver disease or disorder, including adults or juvenile human subjects with such diseases or disorders.

The terms “treating” and “treatment” as used herein refer to reduction in severity and/or frequency of symptoms, elimination of symptoms and/or underlying cause, prevention of the occurrence of symptoms and/or their underlying cause, and/or improvement or remediation of damage. Any liver disorder or disease may be treated using the compositions and methods described herein. Thus, “treating” and “treatment includes:

(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; and/or
(iv) relieving or eliminating the symptoms resulting from the disease or condition, i.e., relieving pain with or without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

A “pharmaceutical composition” refers to a formulation of a compound and/or cells of the disclosure and a medium generally accepted in the art for the delivery of the biologically active compound and/or cells to mammals, e.g., humans. Such a medium includes all pharmaceutically acceptable carriers, diluents or excipients therefor.

“Effective amount” or “amount effective to” refers to that amount of a compound and/or cells which, when administered (e.g., to a mammal, e.g., a human, or mammalian cells, e.g., human cells), is sufficient to effect the indicated outcome (e.g., engraftment, expansion, treatment, etc.). For example, an “effective amount”, such as a “therapeutically effective amount” refers to that amount of a compound and/or cells of the disclosure which, when administered to a mammal, e.g., a human, is sufficient to effect treatment in the mammal, e.g., human. The amount of a composition of the disclosure which constitutes a “therapeutically effective amount” will vary depending on the compound and/or cells, the condition and its severity, the manner of administration, and the age of the mammal to be treated, but can be determined routinely by one of ordinary skill in the art having regard to his own knowledge and to this disclosure.

Ex Vivo Manipulation of Hepatocyte-Generating Cells

Any cell capable of generating a hepatocyte may be subject to ex vivo manipulation (exposure to one or more agents that promote growth, regeneration, survival and/or engraftment) as described herein. Examples of hepatocyte-generating cells include but are not limited to, induced pluripotent stem cells (iPSCs), hepatocyte-like cells (HLCs) for example generated from iPSCs, stem cells, hepatocyte progenitor cells, and/or mature or juvenile hepatocytes.

In certain embodiments, the hepatocyte-generating cells comprise hepatocytes isolated using standard techniques for any source, e.g., from human donors. In certain embodiments, the hepatocytes are primary human hepatocytes (PHH) isolated from screened cadaveric donors, including fresh PHH or cryopreserved PHH.

The hepatocyte-generating cells are thawed, if frozen, and placed in any suitable vessel or culture container. Any suitable culture media can be used. In certain embodiments, the culture medium comprises a Hepatocyte Basal Media, FBS and/or a ROCK inhibitor, for example a 1:1 mix of Hepatocyte Basal Media and Lonza HCM™ Single Quots™, 5% FBS and 10 μM Rho kinase (ROCK) inhibitor. Various hepatocyte-compatible culture media are available, including but not limited to e.g., Liebovitz L-15, minimum essential medium (MEM), DMEM/F-12, RPMI 1640, Waymouth's MB 752/1 Williams Medium E, H 1777, Hepatocyte Thaw Medium (HTM), Cryopreserved Hepatocyte Recovery Medium (CHRM®), Human Hepatocyte Culture Medium (Millipore Sigma), Human Hepatocyte Plating Medium (Millipore Sigma), Human Hepatocyte Thawing Medium (Millipore Sigma), Lonza HCM™, Lonza HBM™, HepatoZYME-SFM (Thermo Fisher Scientific), Cellartis Power Primary HEP Medium (Cellartis), and the like. Various culture supplements and/or substrates may be included or excluded from a desired media, including but not limited to e.g., Lonza Single Quots™ supplements, HepExtend™ Supplement, fetal bovine serum, ROCK inhibitor, dexamethasone, insulin, HEGF, Hydrocortisone, L-glutamine, GlutaMAX™, buffer (e.g., HEPES, sodium bicarbonate buffers, etc.), transferrin, selenium complex, BSA, linoleic acid, collagen, collagenase, Geltrex™, methylcellulose, dimethyl sulfoxide, hyaluronidase, ascorbic acid, antibiotic, and the like. Hepatocyte-compatible media may be general use or specially formulated for primary, secondary, or immortalized hepatocytes and such media may contain serum or growth factors or configured to be serum-free, growth-factor-free, or with minimal/reduced growth factors.

The freshly thawed hepatocyte-generating cells (e.g., human hepatocytes) are then briefly manipulated ex vivo by gently rocking with the presence of one or more agents that promote survival, regeneration and/or engraftment of the hepatocytes. Any molecule(s) involved in hepatocyte regeneration may be targeted, useful reagents include but are not limited to antibodies, and/or nucleic acids (DNA and/or RNA such as mRNAs), and/or small molecules that regulate signaling pathways including but not limited to HGF/c-MET, EGF/EGFR, WNT, TGFβ, HIPPO, Telomere elongation, and the like. Furthermore, any suitable agent(s) can be used in the ex vivo manipulation of hepatocytes as described herein, including but not limited to one or more antibodies or small molecules that target any molecule involved in hepatocyte regeneration, including but not limited to e.g., one or more antibodies or small molecules targeting one or more components the HGF/c-MET signaling pathway, the EGF/EGFR signaling pathway, the WNT signaling pathway, the TGFβ signaling pathway, the HIPPO signaling pathway, telomere elongation, or the like.

In certain embodiments, the agent comprises one or more antibodies, for example an agonist antibody that stimulates hepatocyte survival, growth, regeneration and/or engraftment of the cells (e.g., hepatocytes) as compared to cells/animals not treated as described herein. In some instances, an agonist antibody reagent that stimulates hepatocyte survival, growth, regeneration and/or engraftment by targeting a receptor may have prolonged agonist activity, e.g., as compared to the natural ligand of the receptor. In some instances, agonist antibody activation may persist for a significant period of time after the hepatocytes or hepatocyte-generating cells are separated media containing the agonist antibody, including e.g., after the hepatocytes or hepatocyte-generating cells are transplanted, e.g., into an in vivo bioreactor or a subject. For example, in some instances, pathway activation due to administration of an agonist antibody may persist for 1 or more hours after removal of antibody-containing media, including e.g., 2 or more, 3 or more, 4 or more, 5 or more, 6 or more, 7 or more, 8 or more, 9 or more, 10 or more, 11 or more, or 12 or more hours, or 1 day or more after removal of antibody-containing media. In comparison, pathway activation due to contacting hepatocytes or hepatocyte-generating cells with the natural ligand of the receptor may last only 1 hour or less. Accordingly, in some instances, pathway activation due to an agonist antibody may persist for 2-fold longer or more as compared to pathway activation due to a receptor ligand, including but not limited to e.g., at least 2-fold, at least 3-fold, at least 4-fold, at least 5-fold, at least 6-fold, at least 7-fold, at least 8-fold, at least 9-fold, at least 10-fold, at least 12-fold, at least 14-fold, at least 16-fold, at least 18-fold, or at least 20-fold longer or more compared to pathway activation observed after administration and removal of the corresponding ligand. Pathway activation may be detected and/or measured by a variety of means including but not limited to e.g., upregulation/expression of downstream/effector genes, post-translational modification (e.g., phosphorylation) of one or more pathway components, multimerization (e.g., dimerization), translocation of one or more pathway components, and the like. For example, in some instances, HGF/c-MET pathway activation may be detected and/or measured by analyzing expression of one or more HGF/c-MET downstream effectors or analyzing post-translational modifications due to c-MET activation (such as e.g., tyrosine phosphorylation of GAB1 (pY GAB1). In some instances, EGFR pathway activation may be detected and/or measured by analyzing expression of one or more EGFR downstream effectors or analyzing post-translational modifications due to EGFR activation (such as e.g., tyrosine phosphorylation in the EGFR c-terminal tail).

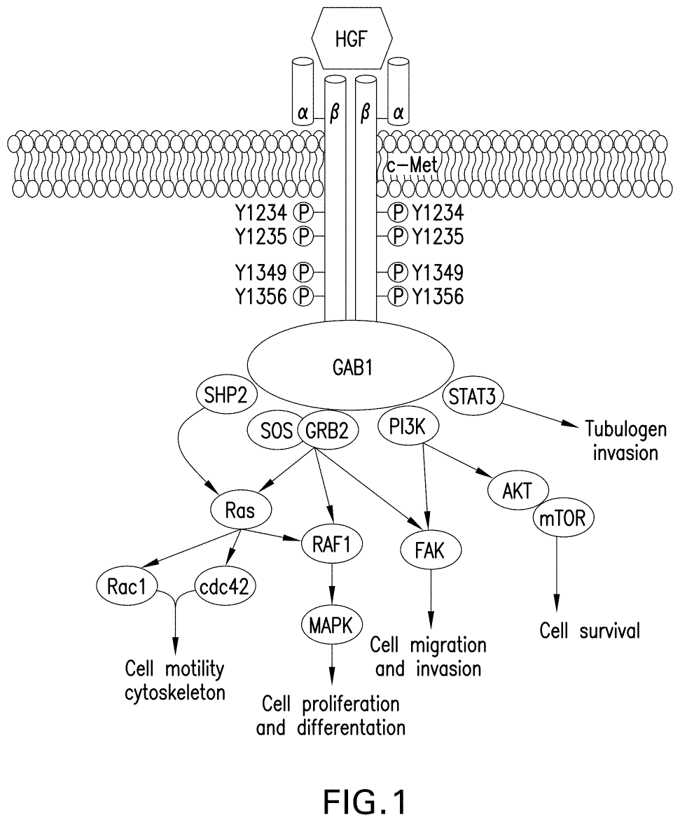

In certain aspects, the one or more antibodies are agonists of HGF/c-MET (a c-MET antibody). As shown in FIG. 1, HGF/c-MET signaling is a key modulator of hepatocyte regeneration and activation of c-MET signaling in hepatocytes induces both pro-survival and pro-proliferation effects downstream. Activation of HGF/c-MET signaling involves ligand binding and dimerization of receptors. Bi-valent monoclonal antibodies against c-MET have been shown to activate this signaling and act as agonists (see, e.g., Ohashi et al. (2000) Nat Med. 6(3):327-31; Yuan et al. (2019) Theranostics 9(7):2115-2128). In addition, while studies have shown that recurring injections of c-MET antibodies in vivo could improve repopulation of transplanted human hepatocytes in mice (see, e.g., Ohashi et al. (2000) Nat Med. 6(3):327-31; Yuan et al. (2019) Theranostics 9(7):2115-2128), it is surprising and unexpected that ex vivo manipulation as described herein enhances hepatocyte repopulation in an animal bioreactor following administration of the cells to the animal. Moreover, it is also surprising and unexpected that the observed enhancement of repopulation persists even in the absence of the c-MET antibody in the animal bioreactor itself (i.e., the observed enhancement in repopulation does not require administration of the agonist to the animal bioreactor). Furthermore, it is also surprising and unexpected that transplantation of ex vivo manipulated hepatocytes as described herein enhances the treatment of subjects with liver disease as compared to transplantation of hepatocytes that have not been manipulated ex vivo as described.

In other embodiments, the agonist antibody targets EGFR. EGFR is a transmembrane tyrosine kinase receptor for ligands including EGF, TGFα, etc. EGFR is highest expressed in hepatocytes of adult liver, plays important role in maintaining liver function, and is indispensable for liver repair and regeneration. Bi-valent monoclonal antibody against EGFR may function as an agonist and activate downstream signaling for cell survival and proliferation. EGFR antibodies are commercially available.

In other embodiments, the agonist antibody target WNT/β-catenin signaling. WNT/β-catenin signaling is involved in a multitude of developmental processes and tissue regeneration by regulating cell proliferation, differentiation, migration and apoptosis. WNT/β-catenin signaling activates when WNT ligand binds to extracellular domain of Frizzled receptor and interacts co-receptor of lipoprotein receptor-related protein (LRP)-5/6. Antibodies against Frizzled or LRP-5/6 which stabilize receptors may function as an agonist antibody and activates the signaling.

Combinations of antibodies may be used. Commercially available antibodies may be used.

One or more different types of agents (e.g., antibodies) may be used in the ex vivo manipulation methods described herein. In certain embodiments, 1, 2, 3, 4, 5, 6, 7, 8, 9 or 10 different antibodies for the same target (e.g., different c-MET antibodies) are used. In other embodiments, one or more antibodies for one target (e.g., c-MET) are used in combination with one or more antibodies for one or more additional targets (e.g., EGFR).

The one or more antibodies and/or small molecules (e.g., agonist antibodies, small molecule agonists) may be specific for one species (e.g., human) or, alternatively, may cross-react with other species (e.g., mouse, rat, pig, etc.). In some embodiments, the agonist antibodies (e.g., c-MET and/or EGFR antibodies) are specific for human c-MET and do not have cross-species activity (e.g., are not cross-reactive with mouse c-MET or EGFR, are not cross-reactive with rat c-MET or EGFR, are not cross-reactive with rodent c-MET or EGFR, are not cross-reactive with pig c-MET or EGFR, are not cross-reactive with other non-human mammal c-MET or EGFR, etc. and combinations thereof). As used herein, “human c-MET specific agonist” and an agonist “specific for human c-MET” refer to agents that specifically bind to human c-MET and specifically activate or enhance human HGF/c-MET signaling (e.g., as measured by phosphorylation of c-MET and/or GAB1 or other readout of pathway activity) without substantially binding to a non-human (e.g., a rodent, pig, etc.) c-MET and/or substantially activating or enhancing non-human HGF/c-MET signaling. As used herein, “human EGFR specific agonist” and an agonist “specific for human EGFR” refer to agents that specifically bind to human EGFR and specifically activate or enhance human EGF/EGFR signaling (e.g., as measured by phosphorylation of EGFR and/or downstream effector activation or other readout of pathway activity) without substantially binding to a non-human (e.g., a rodent, pig, etc.) c-MET and/or substantially activating or enhancing non-human HGF/c-MET signaling.

The one or more antibodies, nucleic acids and/or small molecules may be added to the hepatocyte-generating cells in any way, including but not limited to by addition to the culture media. Additionally, any concentration of the antibodies, nucleic acids and/or small molecules can be used. In some embodiments, antibodies are used at concentrations ranging from 10 ng/mL or less to 1 mg/mL or more, including but not limited to e.g., from 10 ng/mL-1 mg/mL, 25 ng/mL-1 mg/mL, 50 ng/mL-1 mg/mL, 75 ng/mL-1 mg/mL, 100 ng/mL-1 mg/mL, 250 ng/mL-1 mg/mL, 500 ng/mL-1 mg/mL, 750 ng/mL-1 mg/mL, 1 μg/mL-1 mg/mL, 5 μg/mL-1 mg/mL, 10 μg/mL-1 mg/mL, 25 μg/mL-1 mg/mL, 50 μg/mL-1 mg/mL, 75 μg/mL-1 mg/mL, from 10 ng/mL-750 μg/mL, 25 ng/mL-750 μg/mL, 50 ng/mL-750 μg/mL, 75 ng/mL-750 μg/mL, 100 ng/mL-750 μg/mL, 250 ng/mL-750 μg/mL, 500 ng/mL-750 μg/mL, 750 ng/mL-750 μg/mL, 1 μg/mL-750 μg/mL, 5 μg/mL-750 μg/mL, 10 μg/mL-750 μg/mL, 25 μg/mL-750 μg/mL, 50 μg/mL-750 μg/mL, 75 μg/mL-750 μg/mL, from 10 ng/mL-500 μg/mL, 25 ng/mL-500 μg/mL, 50 ng/mL-500 μg/mL, 75 ng/mL-500 μg/mL, 100 ng/mL-500 μg/mL, 250 ng/mL-500 μg/mL, 500 ng/mL-500 μg/mL, 750 ng/mL-500 μg/mL, 1 μg/mL-500 μg/mL, 5 μg/mL-500 μg/mL, 10 μg/mL-500 μg/mL, 25 μg/mL-500 μg/mL, 50 μg/mL-500 μg/mL, 75 μg/mL-500 μg/mL, from 10 ng/mL-250 μg/mL, 25 ng/mL-250 μg/mL, 50 ng/mL-250 μg/mL, 75 ng/mL-250 μg/mL, 100 ng/mL-250 μg/mL, 250 ng/mL-250 μg/mL, 500 ng/mL-250 μg/mL, 750 ng/mL-250 μg/mL, 1 μg/mL-250 μg/mL, 5 μg/mL-250 μg/mL, 10 μg/mL-250 μg/mL, 25 μg/mL-250 μg/mL, 50 μg/mL-250 μg/mL, 75 μg/mL-250 μg/mL, from 10 ng/mL-100 μg/mL, 25 ng/mL-100 μg/mL, 50 ng/mL-100 μg/mL, 75 ng/mL-100 μg/mL, 100 ng/mL-100 μg/mL, 250 ng/mL-100 μg/mL, 500 ng/mL-100 μg/mL, 750 ng/mL-100 μg/mL, 1 μg/mL-100 μg/mL, 5 μg/mL-100 μg/mL, 10 μg/mL-100 μg/mL, 25 μg/mL-100 μg/mL, 50 μg/mL-100 μg/mL, 75 μg/mL-100 μg/mL, 10 ng/mL-75 μg/mL, 10 ng/mL-50 μg/mL, 10 ng/mL-25 μg/mL, 10 ng/mL-10 μg/mL, 10 ng/mL-5 μg/mL, 10 ng/mL-1 μg/mL, 10 ng/mL-750 ng/mL, 10 ng/mL-500 ng/mL, 10 ng/mL-250 ng/mL, 10 ng/mL-100 ng/mL, 10 ng/mL-75 ng/mL, 10 ng/mL-50 ng/mL, 10 ng/mL-25 ng/mL, 50 ng/mL-50 μg/mL, 50 ng/mL-10 μg/mL, 50 ng/mL-5 μg/mL, 50 ng/mL-1 μg/mL, 100 ng/mL-50 μg/mL, 100 ng/mL-10 μg/mL, 100 ng/mL-5 μg/mL, 100 ng/mL-1 μg/mL, 500 ng/mL-50 μg/mL, 500 ng/mL-10 μg/mL, 500 ng/mL-5 μg/mL, 500 ng/mL-1 μg/mL, 1 μg/mL-50 μg/mL, 1 μg/mL-40 μg/mL, 1 μg/mL-30 μg/mL, 1 μg/mL-20 μg/mL, 1 μg/mL-10 μg/mL, 5 μg/mL-50 μg/mL, 5 μg/mL-40 μg/mL, 5 μg/mL-30 μg/mL, 5 μg/mL-20 μg/mL, etc.

In certain embodiments, the hepatocytes (e.g., freshly thawed) are incubated with one or more antibodies (e.g., c-MET and/or EGFR antibodies), which antibody/antibodies are at any effective concentration(s). In certain embodiments, the hepatocyte-generating cells (e.g., freshly thawed human hepatocytes) are incubated with one or more c-MET antibodies, which antibody/antibodies are at a concentration of or about 10 ng/mL or less to 1 mg/mL or more, or any value therebetween, including e.g., those individual values and ranges disclosed herein, including e.g. 10 μg/mL. In certain embodiments, the hepatocyte-generating cells (e.g., freshly thawed human hepatocytes) are incubated with one or more EGFR antibodies, which antibody/antibodies are at a concentration of or about 10 ng/mL or less to 1 mg/mL or more, or any value therebetween, including e.g., those individual values and ranges disclosed herein, including e.g. 10 μg/mL. In other embodiments, the hepatocyte-generating cells (e.g., freshly thawed human hepatocytes) are incubated with one or more c-MET and one or more EGFR antibodies, which antibody/antibodies are at the same or different concentrations, including those concentrations described herein, and where each antibody is at a concentration of or about 10 μg/mL for each antibody type.

Agonistic antibodies employed in ex vivo modulation as described herein may vary in potency and, in some instances, the concentration of antibody employed in an ex vivo modulation may be adjusted accordingly. Useful agonistic antibodies employed in the instant methods may, e.g., have a half maximal effective concentration (EC₅₀) ranging from 0.001 μg/mL or less to 1 μg/mL or more, including but not limited to e.g., 0.001 μg/mL to 1 μg/mL, 0.001 μg/mL to 0.75 μg/mL, 0.001 μg/mL to 0.5 μg/mL, 0.001 μg/mL to 0.25 μg/mL, 0.001 μg/mL to 0.1 μg/mL, 0.001 μg/mL to 0.075 μg/mL, 0.001 μg/mL to 0.05 μg/mL, 0.001 μg/mL to 0.025 μg/mL, 0.005 μg/mL to 1 μg/mL, 0.005 μg/mL to 0.75 μg/mL, 0.005 μg/mL to 0.5 μg/mL, 0.005 μg/mL to 0.25 μg/mL, 0.005 μg/mL to 0.1 μg/mL, 0.005 μg/mL to 0.075 μg/mL, 0.005 μg/mL to 0.05 μg/mL, 0.005 μg/mL to 0.025 μg/mL, 0.01 μg/mL to 1 μg/mL, 0.01 μg/mL to 0.75 μg/mL, 0.01 μg/mL to 0.5 μg/mL, 0.01 μg/mL to 0.25 μg/mL, 0.01 μg/mL to 0.1 μg/mL, 0.01 μg/mL to 0.075 μg/mL, 0.01 μg/mL to 0.05 μg/mL, or 0.01 μg/mL to 0.025 μg/mL. The EC₅₀ of a subject agonistic antibody may be determined by any convenient means, including but not limited to e.g., titration in a flow cytometric binding assay with cells expressing the relevant antigen (e.g., c-MET and/or EGFR) or the like.

The hepatocyte-generating cells and one or more antibodies/small molecules may be incubated together for any period of time (including minutes, hours or days) under any suitable conditions. Incubation times and conditions will vary where useful incubation times will generally be sufficient for activation of the targeted pathway where e.g., the sufficiency of pathway activation may be assessed though the use of any of various readouts of pathway activation, including but not limited to e.g., any such assays described herein. In certain embodiments, the culture is incubated for between 1 to 180 or 240 minutes or more, including e.g., for 15 min., 30 min., 45 min., 1 hour, 2 hours, 3 hours, 15 min. to 4 hours, 30 min. to 4 hours, 45 min. to 4 hours, 1 to 4 hours, 15 min. to 3 hours, 30 min. to 3 hours, 45 min. to 3 hours, 1 to 3 hours, 15 min. to 2.5 hours, 30 min. to 2.5 hours, 45 min. to 2.5 hours, 1 to 2.5 hours, 15 min. to 2 hours, 30 min. to 2 hours, 45 min. to 2 hours, 1 to 2 hours, etc. Incubation may include agitation of the incubating culture where such means of agitation may vary. For example, the hepatocyte-generating cells and one more agents may be contained within a vessel (e.g., a cell culture vessel, a tube, vial, etc.) and the incubating may include various agitation of the vessel, including but not limited to e.g., wherein rocking, shaking, rotation, nutation, and the like.

Hepatocyte Expansion/Repopulation

Following ex vivo manipulation of hepatocyte-generating cells as described herein, the cells, in some instances, are then administered to an animal (e.g., mouse, rat, pig, etc.) for expansion of the hepatocytes in an in vivo bioreactor.

Suitable animal bioreactors for expansion of hepatocytes as described herein are known in the art. In certain embodiments, the animal is genetically modified at one or more loci. Genetic modifications may include knock-out or knock-down to generate an animal that is deficient at one or more loci or activation of one or more target genes. Genetic modifications may be made at multiple loci in any combination (one or more repressive modifications and/or one or more activating modifications). Useful genetic modifications in an in vivo bioreactor may include modifications in various genes including immune genes (e.g., resulting in immunodeficiency), liver function genes (e.g., resulting in liver function deficiency), metabolic genes (e.g., resulting in metabolic deficiency), amino acid catabolism genes (e.g., resulting in deficient amino acid catabolism), and the like.

In certain aspects, the genetically modified animal is a fumarylacetoacetate hydrolase (fah)-deficient animal, for example as described in U.S. Pat. Nos. 8,569,573; 9,000,257 and U.S. Patent Publication No. 20160249591, the disclosures of which are incorporated herein by reference in their entirety. FAH is a metabolic enzyme that catalyzes the last step of tyrosine catabolism. Animals having a homozygous deletion of the Fah gene exhibit altered liver mRNA expression and severe liver dysfunction. Point mutations in the Fah gene have also been shown to cause hepatic failure and postnatal lethality. Humans deficient for Fah develop the liver disease hereditary tyrosinemia type 1 (HT1) and develop liver failure. Fah deficiency leads to accumulation of fumarylacetoacetate, a potent oxidizing agent and this ultimately leads to cell death of hepatocytes deficient for Fah. Thus, Fah-deficient animals can be repopulated with hepatocytes from other species, including humans, containing a functional fah gene. Fah genomic, mRNA and protein sequences for a number of different species are publicly available, such as in the GenBank database (see, for example, Gene ID 29383 (rat Fah); Gene ID 14085 (mouse Fah); Gene ID 610140 (dog FAH); Gene ID 415482 (chicken FAH); Gene ID 100049804 (horse FAH); Gene ID 712716 (rhesus macaque FAH); Gene ID 100408895 (marmoset FAH); Gene ID 100589446 (gibbon FAH); Gene ID 467738 (chimpanzee FAH); and Gene ID 508721 (cow FAH)). Such animals may include a genetically modified fah locus and may or may not include further genetic modifications at other loci, including for example where such an animal (e.g., mouse, pig or rat) is deficient in FAH, RAG-1 or RAG-2, and IL-2Rγ (referred in some instances as an “FRG” animal, such as an FRG mouse, FRG pig, or FRG rat).

Useful genetic modifications also include those resulting in immunodeficiency, e.g., from a lack of a specific molecular or cellular component of the immune system, functionality of a specific molecular or cellular component of the immune system, or the like. In some instances, useful genetic alterations include a genetic alteration of the Recombination activating gene 1 (Rag1) gene. Rag1 is a gene involved in activation of immunoglobulin V(D)J recombination. The RAG1 protein is involved in recognition of the DNA substrate, but stable binding and cleavage activity also requires RAG2. Rag-1-deficient animals have been shown to have no mature B and T lymphocytes. In some instances, useful genetic alterations include a genetic alteration of the Recombination activating gene 2 (Rag2) gene. Rag2 is a gene involved in recombination of immunoglobulin and T cell receptor loci. Animals deficient in the Rag2 gene are unable to undergo V(D)J recombination, resulting in a complete loss of functional T cells and B cells (see e.g., Shinkai et al. Cell 68:855-867, 1992). In some instances, useful genetic alterations include a genetic alteration of the common-gamma chain of the interleukin receptor (Il2rg). Il2rg is a gene encoding the common gamma chain of interleukin receptors. Il2rg is a component of the receptors for a number of interleukins, including IL-2, IL-4, IL-7 and IL-15 (see e.g., Di Santo et al. Proc. Natl. Acad. Sci. U.S.A. 92:377-381, 1995). Animals deficient in Il2rg exhibit a reduction in B cells and T cells and lack natural killer cells. Il2rg is also referred to as interleukin-2 receptor gamma chain.

In some instances, animals may be immunosuppressed, including e.g., where immunosuppression is achieved through administration of one or more immunosuppressive agents. Any suitable immunosuppressive agent or agents effective for achieving immunosuppression in the animal can be used. Examples of immunosuppressive agents include, but are not limited to, FK506, cyclosporin A, fludarabine, mycophenolate, prednisone, rapamycin and azathioprine. Combinations of immunosuppressive agents can also be administered. In some instances, immunosuppressive agents are employed in place of genetic immunodeficiency. In some instances, immunosuppressive agents are employed in combination with genetic immunodeficiency.

As summarized herein, genetically modified animals may include one or more (i.e., a combination of) genetic modifications. For example, such an animal may include a rag1 genetic modification, a rag2 genetic modification, a IL2rg genetic modification, or such an animals may include a rag1 or rag2 genetic modification and a genetic alteration of the Il2rg gene such that the genetic alteration correspondingly results in loss of expression of functional RAG1 protein, RAG2 protein, IL-2rg protein, or RAG-1/RAG-2 protein and IL-2rg protein. In one example, the one or more genetic alterations include a genetic alteration of the Rag2 gene and a genetic alteration of the Il2rg gene. In one example, the one or more genetic alterations include a genetic alteration of the Rag1 gene and a genetic alteration of the Il2rg gene. In some instances, useful genetic alterations include e.g., SCID, NOD, SIRPα, perforin, or nude. Altered loci may be genetic nulls (i.e., knockouts) or other modifications resulting in deficiencies in the gene product at the corresponding loci. Specific cells of the immune system (such as macrophages or NK cells) can also be depleted. Any convenient method of depleting particular cell types may be employed.

It will be appreciated that various models of liver injury, creating a selective growth advantage for hepatocyte xenografts, may be used in the animal bioreactor (e.g., rat, mouse, rabbit, pig) to facilitate hepatocyte engraftment and expansion, including, without limitation, inducible injury, selective embolism, transient ischemia, retrorsine, monocrotoline, thioacetamide, irradiation with gamma rays, carbon tetrachloride, and/or genetic modifications (e.g., Fah disruption, uPA, TK-NOG (Washburn et al., Gastroenterology, 140(4):1334-44, 2011), albumin AFC8, albumin diphtheria toxin, Wilson's Disease, and the like). Combinations of liver injury techniques may also be used.

In some embodiments, the animal is administered a vector (e.g., an Ad vector) encoding a urokinase gene (e.g., urokinase plasminogen activator (uPA)) prior to injection of the heterologous hepatocytes. Expression of uPA in hepatocytes causes hepatic injury and thus permits the selective expansion of hepatocyte xenografts upon transplantation. In one embodiment, the urokinase gene is human urokinase and may be secreted or non-secreted. See, e.g., U.S. Pat. Nos. 8,569,573; 9,000,257 and U.S. Patent Publication No. 20160249591.

In some instances, a TK-NOG liver injury model (i.e., an albumin thymidine kinase transgenic-NOD-SCID-interleukin common gamma chain knockout) may be used as the animal bioreactor as described herein. TK-NOG animals include a herpes simplex virus thymidine kinase hepatotoxic transgene that can be conditionally activated by administration of ganciclovir. Hepatic injury resulting from activation of the transgene during administration of ganciclovir provides a selective advantage to hepatocyte xenografts, facilitating use of such animals as in vivo bioreactors for the expansion of transplanted hepatocytes as described herein.

In some instances, an AFC8 liver injury model (characterized as having a FKBP-Caspase 8 gene driven by the albumin promoter) may be used as the animal bioreactor as described herein. AFC8 animals include a FK508-caspase 8 fusion hepatotoxic transgene that can be conditionally activated by administration of AP20187. Hepatic injury resulting from activation of the transgene during administration of AP20187 provides a selective advantage to hepatocyte xenografts, facilitating use of such animals as in vivo bioreactors for the expansion of transplanted hepatocytes as described herein.

In some instances, an NSG-PiZ liver injury model (characterized as having an α-1 antitrypsin (AAT) deficiency combined with immunodeficiency (NGS)) may be used as the animal bioreactor as described herein. NSG-PiZ animals have impaired secretion of AAT leading to the accumulation of misfolded PiZ mutant AAT protein triggering hepatocyte injury. Such hepatic injury provides a selective advantage to hepatocyte xenografts, facilitating use of such animals as in vivo bioreactors for the expansion of transplanted hepatocytes as described herein. The immunodeficiency renders the animal capable of hosting a xenograft without significant rejection.

In some instances, an animal may be preconditioned prior to receiving a transplantation of hepatocyte-generating cells to improve the recipient livers' ability to support the transplanted cells. Various preconditioning regimens may be employed, including but not limited to e.g., irradiation preconditioning (e.g., partial liver irradiation), embolization preconditioning, ischemic preconditioning, chemical/viral preconditioning (using e.g., uPA, cyclophosphamide, doxorubicin, nitric oxide, retrorsine, monocrotaline, toxic bile salts, carbon tetrachloride, thioacetamide, and the like), liver resection preconditioning, and the like. In some instances, hepatocyte-generating cells may be introduced in the absence of preconditioning and/or a procedure will specifically exclude one, all, or some combination of preconditioning regimens or specific reagents, including e.g., one or more of those described herein. In some instances, induction of liver injury through cessation of NTBC or administration of ganciclovir or AP20187 may be used for preconditioning. Where employed, preconditioning may be performed at some time, including hours, days, or weeks or more, prior to transplantation of hepatocyte-generating cells, including e.g., at least 6 hours, at least 12 hours, at least 24 hours, at least 36 hours, at least 48 hours, at least 60 hours, at least 72 hours, at least 4 days, at least 5 days, at least 6 days, at least a week, or at least two weeks at least prior to transplantation.

After optional pre-conditioning (e.g., with uPA) of the animal (e.g., 24 hours after pre-conditioning), the heterologous hepatocytes can be delivered to the animal via any suitable means. In certain embodiments, the hepatocytes as described herein are administered directly to the liver (e.g., via portal vein injection) and/or via intra-splenic injection where the hepatocytes will travel through the vasculature to reach the liver. In certain embodiments, anywhere between 1×10⁵ and 1×10⁹ (e.g., 5×10⁵/mouse, 10×10⁶/rat, etc.) hepatocytes are introduced into an FRG animal, optionally preconditioned (e.g., 24 hours prior to administration) with adenoviral uPA (e.g., 1.25×10⁹ PFU/25 grams of mouse body weight). The number of hepatocyte-generating cells introduced into the bioreactor will vary and may range, e.g., depending on various factors including the species and size of the animal receiving the cells, from 1×10⁵ or less to 1×10⁹ or more, including but not limited to e.g., 1×10⁵ to 1×10⁹, 1×10⁶ to 1×10⁹, 1×10⁷ to 1×10⁹ 1×10⁸ to 1×10⁹ 1×10⁵ to 1×10⁶ 1×10⁵ to 1×10⁷ 1×10⁵ to 1×10⁸ 1×10⁶ to 1×10⁷ 1×10⁷ to 1×10⁸, 1×10⁶ to 1×10⁸, etc. In some instances, the number of cells administered may be 1×10⁹ or less, including e.g., 0.5×10⁹ or less, 1×10⁸ or less, 0.5×10⁸ or less, 1×10⁷ or less, 0.5×10⁷ or less, 1×10⁶ or less, 0.5×10⁶ or less, 1×10⁵ or less, etc.

In addition, immune suppression drugs can optionally be given to the animals before, during and/or after the transplant to eliminate the host versus graft response in the animal (e.g., the mouse, pig, or rat) from the xenografted heterologous hepatocytes. In some instances, by cycling the animals off immune suppression agents for defined periods of time, the liver cells become quiescent and the engrafted cells will have a proliferative advantage leading to replacement of endogenous hepatocytes (e.g., mouse, pig, or rat hepatocytes) with heterologous hepatocytes (e.g., human hepatocytes). In the case of human hepatocytes, this generates animals with high levels of humanized livers. Heterologous hepatocyte repopulation levels can be determined through various measures, including but not limited to e.g., quantitation of human serum albumin levels, optionally correlated with immunohistochemistry of liver sections from transplanted animals.

In some embodiments, an agent that inhibits, delays, avoids or prevents the development of liver disease is administered to the animal bioreactor during the period of expansion of the administered hepatocytes. Administration of such an agent avoids (or prevents) liver dysfunction and/or death of the animal bioreactor (e.g., mouse, rat, or pig bioreactor) prior to repopulation of the animal bioreactor (e.g., mouse, rat, or pig bioreactor) with healthy (e.g., FAH-expressing) heterologous hepatocytes. The agent can be any compound or composition that inhibits liver disease in the disease model relevant to the bioreactor. One such agent is 2-(2-nitro-4-trifluoro-methyl-benzoyl)-1,3 cyclohexanedione (NTBC), but other pharmacologic inhibitors of phenylpyruvate dioxygenase, such as methyl-NTBC can be used. NTBC is administered to regulate the development of liver disease in a Fah-deficient animal. The dose, dosing schedule and method of administration can be adjusted, and/or cycled, as needed to avoid catastrophic liver dysfunction, while promoting expansion of hepatocyte xenografts, in the Fah-deficient animal bioreactor. In some embodiments, the Fah-deficient animal is administered NTBC for at least two days, at least three days, at least four days, at least five days or at least six days following transplantation of hepatocytes as described herein. In some embodiments, the Fah-deficient animal is further administered NTBC for at least about one week, at least about two weeks, at least about three weeks, at least about four weeks, at least about one month, at least about two months, at least about three months, at least about four months, at least about five months, or at least about six months. In some embodiments, the NTBC (or another compound with a liver protective effect) is withdrawn at about two days, about three days, about four days, about five days, about six days or about seven days following hepatocyte transplantation.

The dose of NTBC administered to the Fah-deficient animal can vary. In some embodiments, the dose is about 0.5 mg/kg to about 30 mg/kg per day, e.g., from about 1 mg/kg to about 25 mg/kg, from about 10 mg/kg per day to about 20 mg/kg per day, or about 20 mg/kg per day. NTBC can be administered by any suitable means, such as, but limited to, in the drinking water, in the food or by injection. In one embodiment, the concentration of NTBC administered in the drinking water is about 1 to about 30 mg/L, e.g., from about 10 to about 25 mg/L, from about 15 to about 20 mg/L, or about 20 mg/L. In certain embodiments, NTBC administration is cyclical from before transplantation to 4 to 8 or more weeks post-transplantation. Furthermore, as using the methods described herein results in 70-90% humanization (repopulation) rates of the human hepatocytes in the animal bioreactor by about 8 weeks, the need for further, potentially harmful, long-term (e.g., 14 days or longer) NTBC withdrawal (i.e., NTBC off) is eliminated.

The animal bioreactor, or subject as described in more detail below, may also be treated with one or more agents as described herein (e.g., a c-MET agonist (e.g., c-MET antibody, small molecule, HGF polypeptide, or derivative thereof), an EGFR agonist (e.g., EGFR antibody, small molecule, EGF polypeptide, or derivative thereof), etc.) before, during and/or after administration of the ex vivo modified hepatocytes. See, e.g., Ohashi et al. (2000) Nat Med. 6(3):327-31; Yuan et al. (2019) Theranostics 9(7):2115-2128. In some instances, a method described herein may specifically exclude administration of one or more agents as described herein (e.g., a c-MET agonist (e.g., c-MET antibody, small molecule, HGF polypeptide, or derivative thereof), an EGFR agonist (e.g., EGFR antibody, small molecule, EGF polypeptide, or derivative thereof), etc.) to an animal bioreactor or subject before, during and/or after administration of ex vivo modified hepatocytes, such that the agent(s) is/are not present in the bioreactor or subject before, during and/or after administration of the ex vivo modified hepatocytes.

Expanded hepatocytes derived from the transplanted hepatocyte-generating cells manipulated as described herein can be collected from the animal bioreactor after any period of time, including but not limited to 7 to 180 days (or any day therebetween) or more after transplantation. In certain embodiments, the expanded hepatocytes are collected 28 to 56 days (or any day therebetween) after transplantation. In some instances, hepatocytes are collected at 1 week, at 2 weeks or earlier, at 3 weeks or earlier, before 4 weeks, at 4 weeks or earlier, at 5 weeks or earlier, at 6 weeks or earlier, at 7 weeks or earlier, before 8 weeks, at 8 weeks or earlier, at 9 weeks or earlier, at 10 weeks or earlier, at 11 weeks or earlier, before 12 weeks, at 12 weeks or earlier, at 13 weeks or earlier, before 14 weeks, or at 14 weeks or earlier.

Furthermore, the expanded hepatocytes can be collected from the animal using any one of a number of techniques. For example, the hepatocytes can be collected by enzymatic digestion of the animal's liver, followed by gentle mincing, filtration, and centrifugation. Furthermore, the hepatocytes can be separated from other cell types, tissue and/or debris using various methods, such as by using an antibody that specifically recognizes the cell type of the engrafted hepatocyte species. Such antibodies include, but are not limited to, an antibody that specifically binds to a class I major histocompatibility antigen, such as anti-human HLA-A, B, C (Markus et al. (1997) Cell Transplantation 6:455-462). Antibody bound hepatocytes can then be separated by panning (which utilizes a monoclonal antibody attached to a solid matrix), fluorescence activated cell sorting (FACS), magnetic bead separation or the like. Alternative methods of collecting hepatocytes may also be employed.

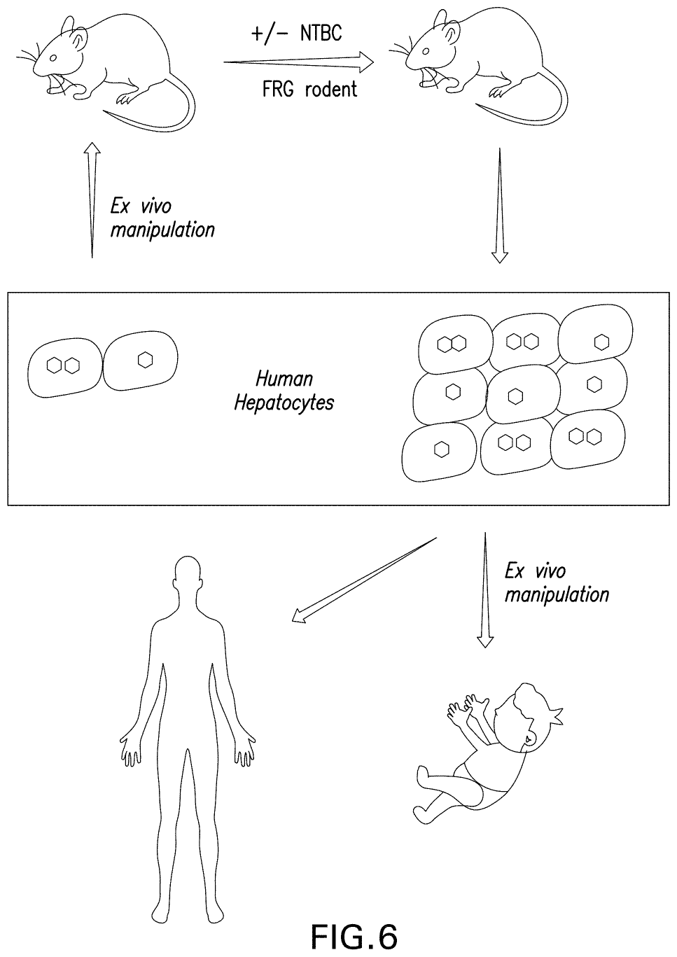

In some instances, collected hepatocytes may be serially transplanted one or more times into additional animal bioreactors. See, e.g., FIG. 6. Serial transplantations may be conducted two, three, four or more times in the same or different species of animal, for example using rats, pigs, mice or rabbits for all serial transplantations or alternatively, using any combination of suitable animal bioreactors for the serial transplantations (one or more in rats, one or more in pigs, etc.).

Furthermore, following collection of the hepatocytes from the animal bioreactor, the hepatocytes may be subject to further ex vivo manipulations (e.g., incubation with one or more agonists, such as agonist antibodies, small molecules, polypeptides, or the like) as described herein prior to administration to a subject. Collected, and optionally isolated, expanded hepatocytes may be used fresh or may be cryopreserved before use.

Compositions

Also described herein are compositions comprising the hepatocyte-generating cells manipulated as described herein as well as hepatocytes generated from these cells.

Thus, provided herein is a live non-human animal (e.g., non-human mammal, rodent, mouse, rat, pig, etc.) comprising a population of hepatocytes (e.g., human hepatocytes) derived (expanded) from hepatocyte-generating cells treated ex vivo as described herein such that more than 40%, more than 50%, more than 60%, more than 70%, more than 80%, or between 80% and 100% of hepatocyte (e.g., human hepatocyte) repopulation rates are achieved over any time period (e.g., 8-16 weeks or longer) in the animal bioreactor by ex vivo manipulated hepatocytes that engraft, survive and expand in the animal. In certain embodiments, more than 70% repopulation is achieved by 8 weeks as compared to current methods in which generally up to 30% repopulation is achieved at the same time period. This greatly improves the health of the animal bioreactor by eliminating weeks of NTBC cycling. In addition, the health, survivability, durability and/or engraftment of repopulated cells derived from transplanted cells treated as described herein is also improved as compared to untreated transplanted cells.

In some instances, provided herein is a non-human in vivo bioreactor (such as a non-human mammal or rodent, e.g., mouse or rat, or pig), or liver thereof, having a hepatocyte population that is, or has been repopulated to, at least 50%, at least 55%, at least 60%, at least 65%, at least 70% or more exogenous (i.e., xenograft-derived) hepatocytes (e.g., human hepatocytes) before 14 weeks, including e.g., at 13 weeks or less, at 12 weeks or less, at 11 weeks or less, at 10 weeks or less, at 9 weeks or less, or at 8 weeks or less following transplantation. Also provided, is a non-human in vivo bioreactor (such as a non-human mammal or rodent, e.g., mouse or rat, or pig), or liver thereof, that includes at least 1×10⁹ exogenous (i.e., xenograft-derived) engrafted and expanded hepatocytes (e.g., human hepatocytes) before 14 weeks, including e.g., at 13 weeks or less, at 12 weeks or less, at 11 weeks or less, at 10 weeks or less, at 9 weeks or less, or at 8 weeks or less post-transplantation. Also provided, is a pig in vivo bioreactor, or liver thereof, that includes at least 30-50×10⁹ exogenous (i.e., xenograft-derived) engrafted and expanded hepatocytes (e.g., human hepatocytes).

In some instances, provided herein is a non-human in vivo bioreactor (such as a non-human mammal or rodent, e.g., mouse or rat, or pig), or liver thereof, having an exogenously-derived (i.e., xenograft) ex vivo manipulated hepatocyte population (e.g., human hepatocyte population) at a time point post-transplantation that is greater than the corresponding exogenously-derived non-ex vivo manipulated hepatocyte population present in a corresponding bioreactor at the same time point post-transplantation. In some instances, the ex vivo manipulated exogenously-derived hepatocyte population is at least 1.1-fold larger, including e.g., at least 1.2-fold, at least 1.3-fold, at least 1.4-fold, at least 1.5-fold, at least 1.6-fold, at least 1.7-fold, at least 1.8-fold, at least 1.9-fold, at least 2-fold, or at least 2.5-fold larger than the corresponding non-ex vivo manipulated exogenously-derived hepatocyte population. In some instances, the ex vivo manipulated exogenously-derived hepatocyte population is at least 10% larger, including e.g., at least 20%, at least 30%, at least 40%, at least 50%, at least 60%, at least 70%, at least 80%, at least 90%, at least 100%, or at least 150% larger than the corresponding non-ex vivo manipulated exogenously-derived hepatocyte population. Such an enhancement in the size of the ex vivo manipulated hepatocyte population, as compared to the corresponding exogenously-derived non-ex vivo manipulated hepatocyte population, may be evaluated at any convenient time point, including e.g., 2 weeks post-transplantation or less or more, including but not limited to at 2 weeks, at 3 weeks, at 4 weeks, at 5 weeks, at 6 weeks, at 7 weeks, at 8 weeks, at 10 weeks, at 12 weeks, at 14 weeks, or at 16 weeks post-transplantation, or any time point therebetween or before or following.

As detailed above, any suitable animal bioreactor may be used for in vivo production of hepatocytes. Non-human mammalian bioreactors are suitable for use. In certain embodiments, the animal is a rodent such as a mouse or rat. In other embodiments, the animal is a pig. The live animal bioreactor may be immunosuppressed/immunocompromised, have undergone liver damage and/or be treated with NTBC (e.g., cycling NTBC treatments) as described above.

In certain embodiments, the compositions comprising hepatocytes as described herein comprise encapsulated hepatocytes. The isolated, expanded hepatocytes may be encapsulated using any method, typically prior to administration to a subject. See, e.g., Jitraruch et al. (2014) PLOS One 9:10; Dhawan et al. (2019) J Hepatol. doi: 10.1016/j.jhep.2019.12.002; Bochenek et al. (2018) Nature Biomedical Engineering 2:810-821. Cell encapsulation within semi-permeable hydrogels represents a local immuno-isolation strategy for cell-based therapies without the need for systemic immunosuppression. The hydrogel sphere facilitates the diffusion of substrates, nutrients, and proteins necessary for cell function while excluding immune cells that would reject the allogeneic cells. Alginate spheres are one of the most widely investigated cell encapsulation materials because this anionic polysaccharide forms a hydrogel in the presence of divalent cations under cell-friendly conditions.

Also provided herein is a decellularized liver, or other acellularized scaffold (including natural and synthetic scaffolds), seeded and/or repopulated with a population of hepatocytes produced by the methods as described herein. For example, a population of ex vivo manipulated hepatocyte-generating cells as described herein may be introduced (with or without other supporting cell types) into a decellularized liver, or portion thereof or other acellularized scaffold, which is subsequently maintained under conditions sufficient for repopulation of the decellularized liver, or portion thereof by hepatocytes generated from the ex vivo manipulated hepatocyte-generating cells.

A liver, such as a human liver or non-human mammal such as a pig, or portion thereof may be obtained, and optionally surgically processed (e.g., to isolate one or more portions or lobe(s) of the liver). The liver, or portion thereof, is then decellularized by any convenient and appropriate means, including e.g., mechanical cell damage, freeze/thawing, cannulation and retrograde profusion of one or more decellularization reagents (e.g., one or more protease (e.g. trypsin), one or more nuclease (e.g., DNase), one or more surfactants (e.g., sodium dodecyl sulfate, Triton X-100, or the like), one or more hypotonic reagents, one or more hypertonic reagents, combinations thereof, or the like. The decellularized liver, or a portion thereof, may be stored and/or presoaked in a hepatocyte-compatible media. Cell suspension containing ex vivo manipulated hepatocyte-generating cells as described herein may then be applied to the decellularized liver, or portion thereof, by any convenient mechanism, such as e.g., injection, perfusion, topical application (e.g., drop-by-drop), or combination thereof. In some instances, the ex vivo manipulated hepatocyte-generating cells may be present in the cell suspension, for seeding into a prepared scaffold, at any convenient and appropriate concentration, including e.g., a concentration of 1×10⁵ or less to 1×10⁷ or more cells per 50 including but not limited to e.g., 1-2×10⁶ cells per 50 μL. Seeded decellularized liver, portions thereof, and/or other acellularized scaffolds may be maintained under suitable conditions for engraftment/attachment and/or expansion of the introduced cells, where such conditions may include suitable humidity, temperature, gas exchange, nutrients, etc. In some instances, a seeded liver, portion thereof, and/or other acellularized scaffold may be maintained in a suitable culture medium a humid environment at or about 37° C. with 5% CO₂. Following attachment and/or expansion of seeded and/or generated hepatocytes to or within the decellularized liver, portion thereof, or other acellularized scaffold, the material may be employed for various uses, including e.g., transplantation into a subject in need thereof, such as a human subject with decreased liver function and/or a liver disease. Methods and reagents relating to decellularization of liver, including human livers, and the production of hepatocyte-receptive acellular scaffolds are described in e.g., Mazza et al. Sci Rep 5, 13079 (2015); Mango et al. Adv. Funct. Mater. 2000097 (2020); Shimoda et al. Sci Rep 9, 1543 (2019); Croce et al. Biomolecules. 2019, 9(12):813; as well as U.S. Pat. No. 10,688,221, the disclosures of which are incorporated herein by reference in their entirety.

Also provided by the present disclosure is a population of hepatocytes produced by the methods as described herein (e.g., a pharmaceutical composition comprising expanded hepatocytes generated as described herein). In certain embodiments, the isolated population of hepatocytes are collected from the animal bioreactor at 10-2000 million human hepatocytes per animal from rodent bioreactor (mouse or rat), including e.g., at least 500 million per rodent, at least 750 million per rodent, at least 1 billion per rodent, etc. In certain embodiments, the isolated population of hepatocytes are collected from the animal bioreactor at 10-50 billion human hepatocytes per animal from a pig bioreactor, including e.g., at least 10 billion per pig, at least 20 billion per pig, at least 30 billion per pig, etc. The isolated populations of expanded hepatocytes as described herein can be used for ex vivo treatment of liver disease in a subject and/or can be further manipulated ex vivo (e.g., via further rounds of the methods described herein) prior to use as an ex vivo treatment for one or more liver conditions.

Populations of hepatocytes produced by the methods as described herein and pharmaceutical compositions thereof may be present in any suitable container (e.g., a culture vessel, tube, flask, vial, cryovial, cryo-bag, etc.) and may be employed (e.g., administered to a subject) using any suitable delivery method and/or device. Such populations of hepatocytes and pharmaceutical compositions may be prepared and/or used fresh or may be cryopreserved. In some instances, populations of hepatocytes and pharmaceutical compositions thereof may be prepared in a “ready-to-use” format, including e.g., where the cells are present in a suitable diluent and/or at a desired deliver concentration (e.g., in unit dosage form) or a concentration that can be readily diluted to a desired delivery concentration (e.g., with a suitable diluent or media). Populations of hepatocytes and pharmaceutical compositions thereof may be prepared in a delivery device or a device compatible with a desired delivery mechanism or the desired route of deliver, such as but not limited to e.g., a syringe, an infusion bag,

Applications

The hepatocytes as described herein can be used for treatment and/or prevention of any liver disease or disorder. For example, reconstitution of liver tissue in a patient by the introduction of hepatocytes is a potential therapeutic option for patients with any liver condition(s) (e.g., acute liver failure, chronic liver disease and/or metabolic or monogenic disease), including as a permanent treatment for these conditions by repopulating the subject's liver with wild-type cells. Hepatocyte reconstitution may be used, for example, to introduce genetically modified hepatocytes for gene therapy or to replace hepatocytes lost as a result of disease, physical or chemical injury, or malignancy. In addition, expanded human hepatocytes can be used to populate artificial liver assist devices. Particular methods of transplanting and expanding heterologous hepatocytes in animals (e.g., rats, mice, rabbits, etc.), as well medical uses of the expanded heterologous hepatocytes are provided herein. Ex vivo manipulated hepatocytes may be administered to a subject in need thereof with or without prior expansion in an in vivo bioreactor.

The methods and compositions described herein can also be applied to expanding hepatocytes after they are transplanted to a human subject. For example, the ex vivo manipulated expanded hepatocytes obtained from animal bioreactors as described herein can be administered to a human subject using known methods (e.g., intravenously). See, e.g., Dhawan et al, Nat Rev Gastroenterol Hepatol, 7:288-98, 2010; Forbes et al, Hepatology, 62: S157-S169, 2015. The transplanted hepatocytes repopulate in the subject more efficiently than hepatocytes produced by other methods. In certain embodiments, repopulation rates of 5-10% or more are achieved in the subject, which is sufficient to be therapeutically effective.

In contrast, in some instances, a method described herein may specifically exclude administration of one or more agents as described herein (e.g., a c-MET agonist (e.g., c-MET antibody, c-MET agonist small molecule, HGF polypeptide, or derivative thereof), an EGFR agonist (e.g., EGFR antibody, EGFR agonist small molecule, EGF polypeptide, or derivative thereof), etc.) to a subject before, during and/or after administration of ex vivo modified hepatocytes (whether or not such hepatocytes are first expanded in an in vivo bioreactor), such that the agent(s) is/are not present in the subject before, during and/or after administration of the ex vivo modified hepatocytes. Accordingly, methods described herein include treatments where the subject is not, at any point during the treatment, administered the reagent used during ex vivo manipulation of the hepatocytes.

The compositions and methods described herein provide a novel method of treating and/or preventing liver disease in a human subject as the ex vivo expanded hepatocytes provided herein are the first hepatocytes produced in animal bioreactor that can be used directly for therapy. This surprising and unexpected stand-alone use is a result of the significantly increased expansion and/or engraftment of the ex vivo manipulated hepatocytes in the animal bioreactor and/or their increased expansion and/or engraftment potential upon transplantation into a patient. Thus, the methods described herein can be used for hepatocyte cell therapy in clinic by providing healthy hepatocytes, including as a stand-alone therapy, which, due to the enhanced engraftment profile results in more efficient disease treatment and/or prevention than current methods.

Hepatocytes as described herein and compositions comprising hepatocytes as described herein can be administered to subjects by any suitable means and to any part, organ, tissue or the subject. Non-limiting examples of administration means include portal vein infusion, umbilical vein infusion, direct splenic capsule injection, splenic artery infusion, infusion into the omental bursa and/or intraperitoneal injection (infusion, transplantation). In certain embodiments, the compositions comprise encapsulated hepatocytes that are transplanted by infusion into the intraperitoneal space and/or the omental bursa. In certain embodiments, the compositions comprise acellular/decellularized scaffold, including e.g., synthetic scaffolds, decellularized liver, and the like, that are seeded and/or repopulated with hepatocytes as described herein and surgically transplanted into a subject in need thereof.

Prior and/or after administration of the hepatocytes as described herein, the patient may also be treated with one or more agents (e.g., antibodies, small molecules, RNA, etc.) that promote growth, regeneration, survival and/or engraftment of hepatocytes in the subject. In certain embodiments, the patient may be treated with at least one c-MET antibody, optionally one that human-specific. The one or more agents may be administered to the patient 1, 2, 3, 4, 5 or more times and may be administered with and/or at different times than the hepatocytes. In some instances, prior and/or after administration of the hepatocytes as described herein, the patient may not be treated with one or more, or any additional, agents (e.g., antibodies, small molecules, RNA, etc.) that promote growth, regeneration, survival and/or engraftment of hepatocytes in the subject. Accordingly, in some instances, the administered hepatocytes may be the sole active agent administered to the subject to treat the subject for the condition.

In addition to or as an alternative to administration (transplantation) to a subject (patient), the hepatocytes as described herein can be also be used for supplying hepatocytes to devices or compositions useful in treating subjects with liver disease. Non-limiting examples of such devices or compositions in which the hepatocytes of the present disclosure can be used include bioartificial livers (BAL) (extracorporeal supportive devices for subjects suffering from acute liver failure) and/or decellularized livers (recellularizing organ scaffolds to provide liver function in the subject). See, e.g., Shaheen et al. (2019) Nat Biomed Eng. doi: 10.1038/s41551-019-0460-x; Glorioso et al. (2015) J Hepatol 63(2):388-98.

Furthermore, any of the ex vivo methods involving administration of hepatocytes to a subject may further comprise repeating one or more steps of the methods, including for example repeated administration of the hepatocytes and/or agents as described herein at any time.

Disease and disorders that can be treated by the methods and compositions described herein include but are not limited to Crigler-Najjar syndrome type 1; familial hypercholesterolemia; Factor VII deficiency; Glycogen storage disease type I; infantile Refsum's disease; Progressive familial intrahepatic cholestasis type 2; hereditary tyrosinemia type 1; and various urea cycle defects; acute liver failure, including juvenile and adult patients with acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including cirrhosis; acute-on-chronic liver disease caused by one of the following acute events: alcohol consumption, drug ingestion, and/or hepatitis B flares. Thus, the patients may have one or more of these or other liver conditions.

In some instances, diseases and disorders treated according to the methods described herein may include hepatocyte-specific (hepatocyte-intrinsic) dysfunction. For example, the dysfunction, and the etiology of the disease and/or disorder, may be due to, or primarily attributable to, dysfunction of the endogenous hepatocytes present within the subject. In some instances, the hepatocyte-specific dysfunction may be genetic or inherited by the subject. In some instances, the etiology of the disease or disorder does not substantially involve cell types other than hepatocytes. In some instances, the disease or disorder results in decreased liver function, liver disease (acute or chronic), or other adverse condition derived from the endogenous hepatocytes. Accordingly, in some instances, e.g., where a disease is intrinsic to the endogenous hepatocyte population, an effective treatment may include replacement, supplementation, transplantation, or repopulation with hepatocytes as described herein. Without being bound by theory, in hepatocyte-intrinsic diseases/disorders replacement and/or supplementation of the endogenous hepatocytes can result in significant clinical improvement without the disease/disorder negatively impacting the transplanted hepatocytes. For example, where a subject has a genetic disorder affecting hepatocyte function (e.g., amino acid metabolism within hepatocytes, such as e.g., a hypertyrosinemia) allogenic transplanted hepatocytes may be essentially unaffected by the presence of the disease/disorder within the subject. Thus, transplanted hepatocytes may substantially engraft, survive, expand, and/or repopulate within the subject, resulting in a significant positive clinical outcome.

Diseases and disorders characterized by hepatocyte-specific (hepatocyte-intrinsic) dysfunction may be contrasted with diseases and disorders having an etiology that is not hepatocyte specific and involve hepatocyte extrinsic factors. Examples of diseases having factors and/or an etiology that is hepatocyte extrinsic include but are not limited to e.g., alcoholic steatohepatitis, alcoholic liver disease (ALD), hepatic steatosis/nonalcoholic fatty liver disease (NAFLD), and the like. Hepatocyte extrinsic diseases involve hepatic insults that are external, or derived from outside the endogenous hepatocytes, such as alcohol, diet, infection, etc.

Examples of hepatocyte-intrinsic and hepatocyte-related diseases include liver-related enzyme deficiencies, hepatocyte-related transport diseases, and the like. Such liver-related deficiencies may be acquired or inherited diseases and may include metabolic diseases (such as e.g. liver-based metabolic disorders). Inherited liver-based metabolic disorders may be referred to “inherited metabolic diseases of the liver”, such as but not limited to e.g., those diseases described in Ishak, Clin Liver Dis (2002) 6:455-479. Liver-related deficiencies may, in some instances, result in acute and/or chronic liver disease, including e.g., where acute and/or chronic liver disease is a result of the deficiency when left untreated or insufficiently treated. Non-limiting examples of inherited liver-related enzyme deficiencies, hepatocyte-related transport diseases, and the like include Crigler-Najjar syndrome type 1; familial hypercholesterolemia, Factor VII deficiency, Glycogen storage disease type I, infantile Refsum's disease, Progressive familial intrahepatic cholestasis type 2, hereditary tyrosinemias (e.g., hereditary tyrosinemia type 1), genetic urea cycle defects, phenylketonuria (PKU), hereditary hemochromatosis, Alpha-I antitrypsin deficiency (AATD), Wilson Disease, and the like. Non-limiting examples of inherited metabolic diseases of the liver, including metabolic diseases having at least some liver phenotype, pathology, and/or liver-related symptom(s), include 5-beta-reductase deficiency, AACT deficiency, Aarskog syndrome, abetalipoproteinemia, adrenal leukodystrophy, Alpers disease, Alpers syndrome, alpha-1-antitrypsin deficiency, antithrombin III deficiency, arginase deficiency, argininosuccinic aciduria, arteriohepatic dysplasia, autoimmune lymphoproliferative syndrome, benign recurrent cholestasis, beta-thalassemia, Bloom syndrome, Budd-Chiari syndrome, carbohydrate-deficient glycoprotein syndrome, ceramidase deficiency, ceroid lipofuscinosis, cholesterol ester storage disease, cholesteryl ester storage disease, chronic granulomatous, chronic hepatitis C, Crigler-Najjar syndrome, cystic fibrosis, cystinosis, diabetes mellitus, Dubin-Johnson syndrome, endemic Tyrolean cirrhosis, erythropoietic protoporphyria, Fabry disease, familial hypercholesterolemia, familial steatohepatitis, fibrinogen storage disease, galactosemia, gangliosidosis, Gaucher disease, genetic hemochromatosis, glycogenosis type 1a, glycogenosis type 2, glycogenosis type 3, glycogenosis type 4, granulomatous disease, hepatic familial amyloidosis, hereditary fructose intolerance, hereditary spherocytosis, Hermansky-Pudlak syndrome, homocystinuria, hyperoxaluria, hypobetalipoproteinemia, hypofibrinogenemia, intrahepatic cholestasis of pregnancy, Lafora disease, lipoamide dehydrogenase deficiency, lipoprotein disorders, Mauriac syndrome, metachromatic leukodystrophy, mitochondrial cytopathies, Navajo neurohepatopathy, Niemann-Pick disease, nonsyndromic paucity of bile ducts, North American Indian childhood cirrhosis, ornithine transcarbamylase deficiency, partial lipodystrophy, Pearson syndrome, porphyria cutanea tarda, progressive familial intrahepatic cholestasis, progressive familial intrahepatic cholestasis type 1, progressive familial intrahepatic cholestasis type 2, protein C deficiency, Shwachman syndrome, Tangier disease, thrombocytopenic purpura, total lipodystrophy, type 1 glycogenosis, Tyrolean cirrhosis, tyrosinemia, urea cycle disorders, venocclusive disease, Wilson disease, Wolman disease, X-linked hyper-IgM syndrome, and Zellweger syndrome,

Treatment of subjects according to the methods described herein may result in various clinical benefits and/or measurable outcomes, including but not limited to e.g., prolonged survival, delayed disease progression (e.g., delayed liver failure), prevention of liver failure, improved and/or normalized liver function, improved and/or normalized amino acid levels, improved and/or normalized ammonia levels, improved and/or normalized albumin levels, improved and/or normalized bilirubin, recovery from a failure to thrive phenotype, reduction in lethargy, reduction in obtundation, reduction in seizures, reduction in jaundice, improved and/or normalized serum glucose, improved and/or normalized INR, improved and/or normalized urine test results, and the like. For example, in some instances, administration of hepatocyte-generating cells, such as hepatocytes, that have been ex vivo manipulated as described herein results in at least a 5% increase in survival of subjects having a liver disease and/or a condition resulting in liver failure as compared to e.g., subjects treated according to the standard of care and/or administered hepatocyte-generating cells that have not been ex vivo manipulated as described herein. The observed level of enhanced survival in such subject may vary and may range from an at least 5% to 60% or more increase, including but not limited to e.g., an at least 5%, at least 10%, at least 15%, at least 20%, at least 25%, at least 30%, at least 35%, at least 40%, at least 45%, at least 50%, at least 55%, at least 60% or more increase in survival. In some instances, subjects administered hepatocyte-generating cells that have been ex vivo manipulated as described herein may experience a delay in disease progression and/or the onset of one or more disease symptoms, such as but not limited to e.g., liver failure and/or any symptom(s) attributable thereto. Such a delay in disease progression and/or symptom onset may last days, weeks, months or years, including but not limited to e.g., at least one week, at least one month, at least 2 months, at least 3 months, at least 4 months, at least 5 months, at least 6 months, at least a year or more. The hepatocytes as described herein administered to a patient effect a beneficial therapeutic response in the patient over time.

The following Examples relate to exemplary embodiments of the present disclosure. It will be appreciated that this is for purposes of exemplification only and that other antibodies, nucleic acids (e.g., DNA and/or RNA) or small molecules (other than c-MET) can also be used.

### EXAMPLES

### Example 1: Characterization of c-MET Antibodies

Commercially obtained c-MET antibodies were evaluated in vitro for signaling activation in HepG2 and in primary human hepatocytes (PHH). In particular, cells were incubated with commercially obtained antibodies for 2 hours under standard conditions and evaluated by FACS analysis and Western Blot. Antibodies which recognize native human c-MET receptors by FACS and activate the HGF/c-MET signaling pathway in human liver cells were characterized as c-MET agonist antibodies.

In addition, antibody kinetics were evaluated by a wash-out assay as follows. HepG2 cells were agonized with or without c-MET antibodies (10 μg/mL) (or HGF control (100 ng/mL)) for 1 hour. The antibodies were retained in the sample or washed out and samples taken at the following timepoints: 1 hour, 2.5 hours, 5 hours, 1 day, 2 days and 5 days post treatment.

Results showed that treatment with c-MET agonist antibodies or HGF (100 ng/mL) for 1 hour highly activated the c-MET/GAB1 signaling pathway. Furthermore, it was unexpectedly found that, in both the agonist-retained and agonist-washed-out conditions, the signaling activation due to treatment with c-MET antibodies was significantly more durable over time (e.g., up to 5 days for retained samples and 2 days for washed-out samples) than the signaling activation seen in samples treated with HGF.

These results demonstrate that c-MET agonistic antibody treatment can provide prolonged and more sustained pathway activation (e.g., as compared HGF-induced pathway activation), both when the respective agonist remains in culture with the cells and when washed-out/removed after the initial incubation time.

### Example 2: Ex Vivo Manipulation of Hepatocytes

Primary human hepatocytes were manipulated ex vivo prior to transplantation into FRG animals and the effect of c-MET antibody ex vivo manipulation on expansion and engraftment of the transplanted hepatocytes was evaluated as follows.

Primary hepatocytes were obtained from BD and stored at −80° C. Hepatocyte media was made as follows: 1:1 mix of Hepatocyte Basal Media (Lonza) and HCM Single™ Quots™, 5% FBS and 10 uM ROCK inhibitor. For these experiments, c-MET antibodies were obtained commercially from Sino Biological (c-MET Ab #1) and R&D Systems (c-MET Ab #2). EGFR antibody was obtained commercially from Sino Biological.

On the day of transplantation (day 0), cryopreserved primary human hepatocytes are thawed and prepared according to the following protocol:
(1) Warm 1×50 ml Hepatocyte Thaw Media (Thermo) to 37° C. Quickly thaw cryopreserved human hepatocytes in 37° C. water bath and transfer hepatocytes to Hepatocyte Thawing Media (Thermo)
(2) Centrifuge cell suspension at 100 g for 10 min at RT to form cell pellet and then discard supernatant.
(3) Gently re-suspend cell pellet by swirling and then add 47 ml hepatocyte media.
(4) Centrifuge cell suspension at 80 g for 4 min at RT to form cell pellet and then discard supernatant.
(5) Gently re-suspend cell pellet by swirling in a small volume of hepatocyte media (various by cell lot, to an estimated cell density of 1.0-2.0×10⁶
(6) Perform manual cell counting on hemocytometer with trypan blue staining, to determine numbers of viable and dead hepatocytes.
(7) Adjust concentration of viable hepatocytes to 1.0×10⁶ cells/ml in hepatocyte media.
(8) Mix cells and desired concentration of antibodies for each group and plate cells to 6-well ultra-low attachment plates at 2 ml/well (cell density 1.0×10⁶ cell/ml). Place plates on a rocking platform inside the incubator and rock for 1-2 hours.
(9) Manual gentle shaking/mixing every 30 min during the rocking process to mix.
(10) After rocking, transfer cells to 15 ml tubes.
(11) Spin down at 80 g for 4 mins.
(12) Aspirate supernatant (removal of unbound antibodies).
(13) Gently resuspend hepatocytes in hepatocyte media with DNase (2 ug/ml) in 100 ul aliquots per animal transplantation, placing each aliquot into an individual tube for each transplantation. Keep cells on ice until transplantation.

### Example 3: Production of Hepatocytes in In Vivo Bioreactors

Human hepatocytes prepared as described above in Example 2 were transplanted into FRG mice through intrasplenic injection following a standard transplantation protocol. Mice were cycled on/off NTBC per the NTBC cycling regimen as described in U.S. Pat. No. 8,569,573, the disclosure of which is incorporated herein by reference in its entirety.

Livers were harvested at 1, 4, and 8 weeks after transplantation and repopulation of transplanted human hepatocytes was evaluated by FAH IHC and human albumin ELISA as described in U.S. Pat. No. 8,569,573, the disclosure of which is incorporated herein by reference in its entirety.

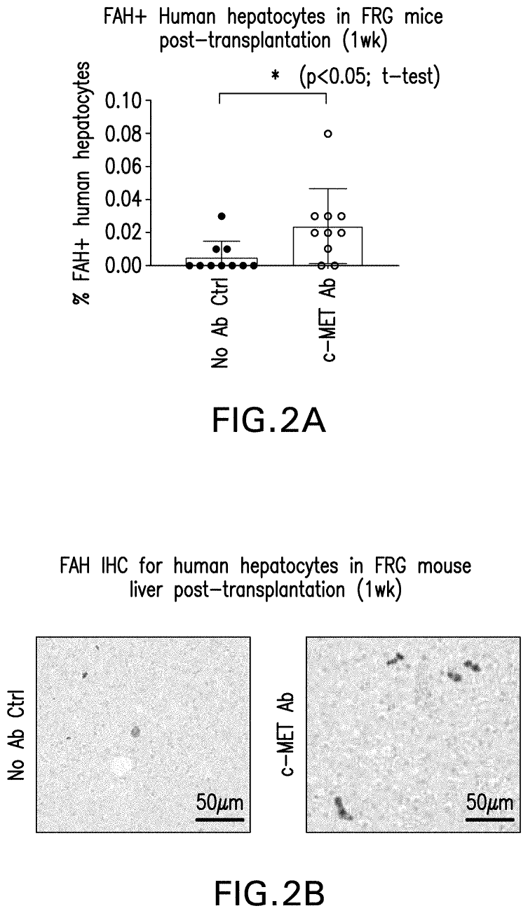

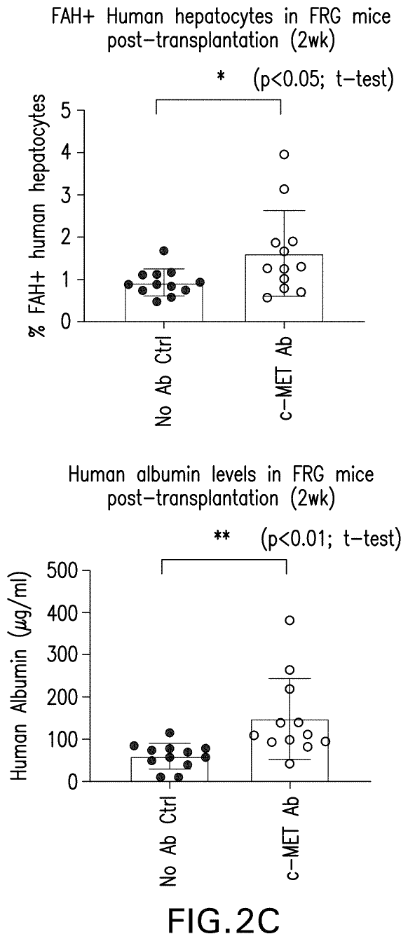

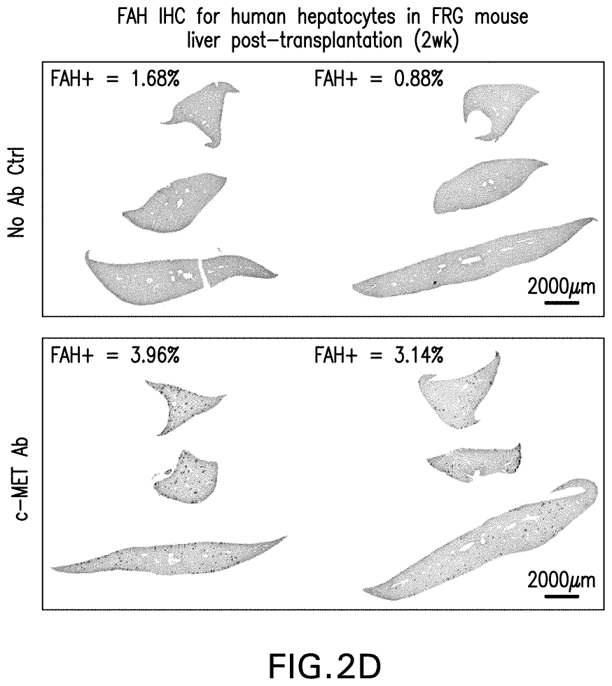

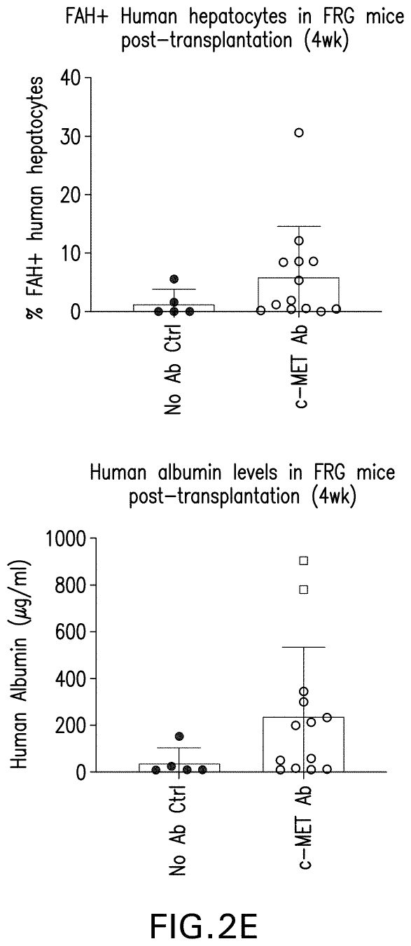

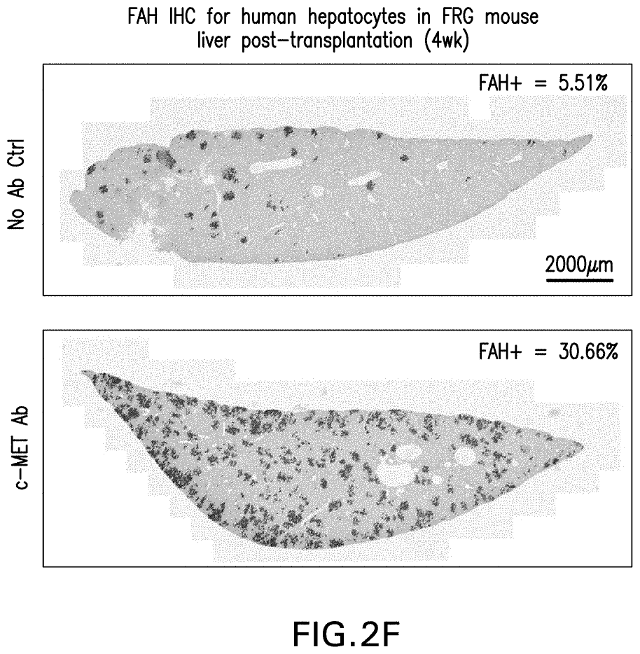

As shown in FIG. 2A through FIG. 5, ex vivo manipulation of hepatocytes led to increased levels of engraftment and expansion in FRG animals. In particular, ex vivo manipulation with c-MET agonist antibodies dramatically improved the in vivo repopulation kinetics of transplanted human hepatocytes by reaching 70-90% repopulation in 8 weeks as compared to the 5-30% repopulation range obtained using current procedures (i.e., procedures lacking ex vivo manipulation as described herein).

FIGS. 2A and 2B demonstrate, using qualitative (FIG. 2B) and quantitative (FIG. 2A) assessment by FAH IHC, increased engraftment and expansion at 1 week post-transplantation in animals that received hepatocytes manipulated ex vivo by application of agonistic c-MET antibody (“c-MET Ab”), as compared to animals that received hepatocytes that were not subjected to ex vivo manipulation (“No Ab Ctrl”). FIG. 2C and FIG. 2D similarly demonstrate increased hepatocyte repopulation at 2 weeks post-transplantation in animals that received ex vivo manipulated hepatocytes as compared to animals that received hepatocytes that were not ex vivo manipulated. In particular, these results show, not only increased numbers of hepatocytes in the c-MET Ab group by FAH IHC (FIG. 2C, top graph, and FIG. 2D), but also enhanced functional repopulation as measured by higher human albumin levels in the c-MET Ab group as compared to control (FIG. 2C, bottom graph). FIG. 2E and FIG. 2F further demonstrate continued enhancement of repopulation at 4 weeks post-transplantation in animals that received hepatocytes that were ex vivo manipulated with c-MET agonist antibody as compared to control, as measured by FAH IHC (FIG. 2E, top graph, and FIG. 2F) and human albumin ELISA (FIG. 2E, bottom graph). Additional studies, quantified by human albumin ELISA at 4 and 6 weeks post-transplantation, further demonstrated, on average, about a 2-fold increase or greater in repopulation rates in mice that received treated hepatocytes manipulated ex vivo with c-MET agonist antibody as compared to animals that received untreated, control (i.e., non-ex vivo manipulated) hepatocytes. For example, such further studies showed mean human albumin levels, at 4 weeks post-transplantation, of 388 μg/mL in mice that received c-Met agonist antibody ex vivo manipulated hepatocytes as compared to 58 μg/mL in controls that received hepatocytes not subjected to the ex vivo manipulation, a difference that was statistically significant (p=0.0076).

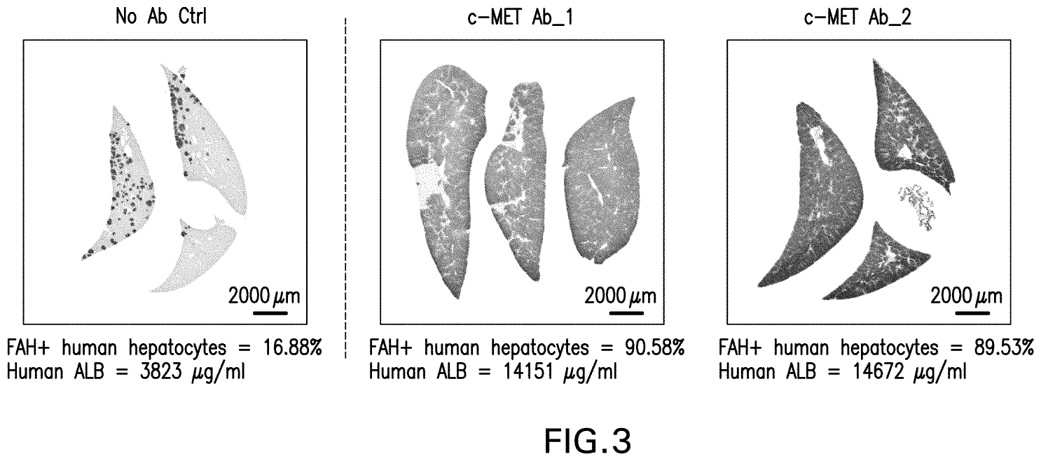

Exemplary results shown in FIG. 3 demonstrate that, at 8 weeks after transplantation, a control animal bioreactor that received a transplantation of untreated human hepatocytes had less than 17% repopulation of FAH+ human hepatocytes and the human albumin level in this animal was less than 4000 μg/mL (left panel, “No Ab Ctrl”). By contrast, ˜90% levels of FAH+ human hepatocyte repopulation were achieved in animals transplanted with human hepatocytes treated with c-MET agonist antibodies (middle (“c-MET Ab_1”) and right (“c-MET Ab_2”) panels). In addition, human albumin levels above 14,000 μg/mL were observed in these ex vivo manipulated animals. In further studies, quantification at 8 weeks post-transplantation by FAH liver IHC and blood human albumin ELISA showed repopulation levels above 70% and human albumin levels above 4000 μg/mL in multiple animals that received c-MET agonist treated hepatocytes. On average, repopulation (e.g., as measured by FAH liver IHC and/or blood human albumin ELISA) was enhanced by about two-fold or more at 8 weeks post-transplantation in animals that received c-MET agonist antibody ex vivo manipulated hepatocytes as compared to animals that received hepatocytes that were not manipulated ex vivo with c-MET agonist antibody.

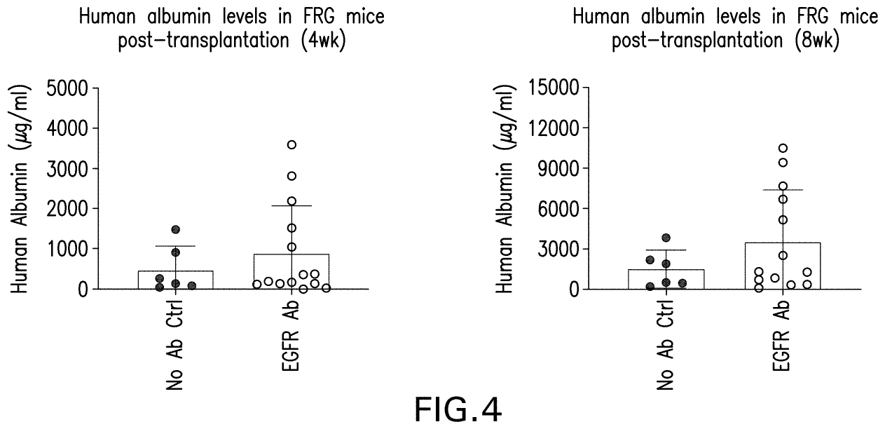

As shown in FIG. 4, ex vivo manipulation of cells with EGFR antibodies also improved repopulation as compared to untreated cells at both 4 weeks and 8 weeks post-transplantation. In a separate study, enhanced levels of repopulation were also observed as early as 2 weeks post-transplantation in mice that received EGFR antibody ex vivo manipulated human hepatocytes as compared to control mice that received human hepatocytes that had not been ex vivo manipulated. In particular, mice with human albumin levels above 100 μg/mL were detected at 2 weeks in the EGFR antibody ex vivo manipulated group and the mean of this group is >2 fold higher compared to the non-ex vivo manipulated group in which all animals had human albumin levels below 50 μg/mL.

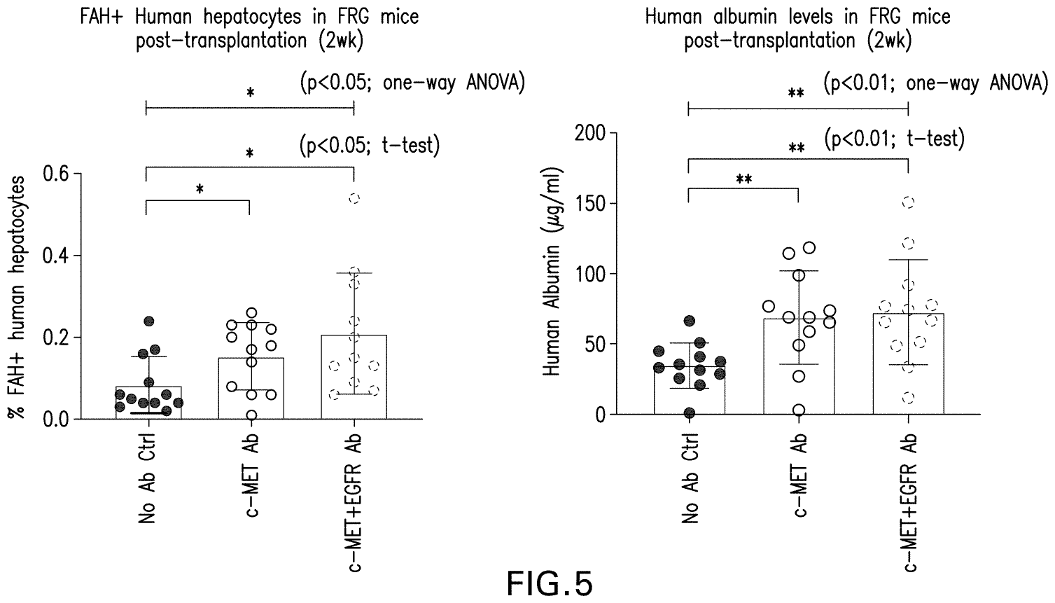

As shown in FIG. 5, cells treated with the c-MET+EGFR antibodies prior to transplantation also significantly increased engraftment and expansion of human hepatocytes in the animal bioreactor as compared to untreated cells as determined by albumin production levels and FAH IHC at 2 weeks. In addition, both maximum and mean repopulation by cells treated ex vivo with both c-MET and EGFR antibodies were greater at 2 weeks than the corresponding repopulation levels observed in animals that received cells treated ex vivo with c-MET antibody alone.

Rat FRG animals have also been used as in vivo bioreactors for the production (i.e., expansion) of hepatocytes (e.g., human hepatocytes). In such methods, human hepatocytes are treated as described above in Example 2 and are administered to rats cycled on/off NTBC (e.g., similar to the NTBC cycling as described above for mice). Human hepatocytes, including primary human hepatocytes, may be manipulated ex vivo by contact with at least one agent that promotes growth, regeneration, survival and/or engraftment of hepatocytes and transplanting, including, e.g., a c-MET agonist (such as a c-MET agonist antibody), an EGFR agonist (such as an EGFR agonist antibody), and the like. Rat livers are harvested 2, 4, 8, 12 and/or 16 weeks post-transplantation and evaluated for repopulation by the transplanted hepatocytes. For example, the harvested rat livers may be evaluated for human protein expression, such as human FAH expression, as described above. In some instances, blood samples may be obtained from live rats for in-study evaluation of repopulation, e.g., through the use of human albumin quantification as a surrogate measure of the level of repopulation by transplanted cells. Optionally, rats are also treated with c-MET and/or EGFR antibodies one or more time before, during and/or after transplantation.

Ex vivo manipulation, including exposure to c-MET antibodies, increases levels of engraftment and expansion in FRG rats, achieving at least 50-70% or more repopulation by 8-16 weeks post-transplantation.

FAH IHC quantification of human hepatocyte repopulation in FRG rodent model, as described herein, was performed as follows. IHC slides stained for FAH positive cells (by a FAH specific antibody) were scanned by the Pannoramic Midi II slide scanner. The scanned slides were then analyzed using CaseViewer software, CellQuant module. A standard scenario was built under the module properties and measurement parameters. A cell was defined by the width of the cytoplasm and the stain intensity of the cytoplasm. Cell detection was done through color deconvolution, chromogen indicating positivity and counterstain indicating negativity in the cell cytoplasm. A FAH positive cell was defined by the staining intensity (0, +1, +2 or +3) where 0 is no positive intensity detected and +3 is strong positive intensity detected. Scoring was adjusted where necessary. The repopulation rate was determined as the percentage of cells +3 (strong FAH positive) versus total cells detected (based on the cell detection criteria described above).

### Example 4: Enhanced Rescue of Liver Disease by Transplantation of Ex Vivo Manipulated Human Hepatocytes

FRG rats were used in this study as a clinically relevant model of liver disease as, in the absence of NTBC, such rats recapitulate liver failure which is observed to result from untreated type 1 hereditary tyrosinemia in human patients. Correspondingly, modeling the human disease, in the absence of an alternative intervention FRG rats develop, and ultimately die of, liver failure. To test the ability of ex vivo manipulated human hepatocytes, as described herein, to treat liver failure in vivo in this model, FRG rats were administered either a cell therapy dose of (1) primary human hepatocytes manipulated ex vivo with c-MET antibody agonist or (2) control primary human hepatocytes that were not manipulated ex vivo with the c-MET antibody agonist. Transplanted animals were maintained without NTBC supplementation throughout the course of observation described herein and animal survival was assayed as a marker of disease progression. By 7 days post-transplantation a survival rate of 91.7% was observed in the group of rats that received c-MET agonist antibody ex vivo manipulated hepatocytes as compared to 25% survival in the group treated with hepatocytes that were not ex vivo manipulated. This study demonstrates that administration of the ex vivo manipulated human hepatocytes described herein effectively treats liver failure in a rodent model of human disease. These data show that administration of the ex vivo manipulated human hepatocytes enhanced survival, and delayed disease progression, as compared to a matched control treatment that included hepatocytes not subjected to ex vivo manipulation as described herein.

### Example 5: Ex Vivo Therapy

Pharmaceutical compositions comprising human hepatocytes prepared in animal bioreactors as described herein, for instance as in Example 3, are transplanted into human subjects with one or more liver diseases or disorders using standard protocols.

In some instances, prior to transplantation, the hepatocytes isolated from the animal bioreactors may be encapsulated using standard techniques, for example as follows. Empty and hepatocyte microbeads (EMBs and HMBs) are produced essentially as described in Dhawan et al. (2019) J Hepatol. 72(5):P877-884 and Jitraruch et al. (2014) PLOS One 9:10. Briefly, hepatocyte microbeads are produced using the IE-50R encapsulator (Inotech Encapsulation AG, Dottikon, Switzerland) with a 250-μm nozzle and sterile clinical grade reagents. Ultrapure sodium alginate, with low-viscosity and high-glucuronate (PRONOVA™ SLG20; NovaMatrix, Sandvika, Norway) dissolved in 0.9% NaCl to give a final concentration of 1.5% alginate solution (w/v), and mixed with cells at the density of 2.5×10⁶ cell/ml alginate. Microbeads are cross-linked in 1.2% CaCl₂) solution for 10 min and washed twice with 0.9% NaCl to remove excess Ca′ ions. The microbeads mean diameter is 500 SD 100 μm.

Hepatocyte compositions (including alginate HMBs) are administered to the subject. Administration can be via infusion into the intraperitoneal cavity, including in the intensive care unit under continuous cardiorespiratory monitoring. Subjects (adults and juveniles) may be ventilated as part of the management of acute liver failure at the time of infusion. Prior to treatment, international normalized ratio (INR) is corrected to <2 and platelets >50,000/microliter. A 16-gauge cannula is placed under ultrasound guidance through the anterior abdominal wall and between 5-20 ml/kg/session of hepatocytes (e.g., alginate HMBs in cell media are infused over 20-45 minutes under ultrasound guidance). The dose may be calculated to approximately 25 million cells per ml alginate. Patients are generally monitored for vital signs, abdominal distention, intestinal ileus, bleeding in the abdomen, urine output and/or signs of anaphylaxis or infection.

The transplanted hepatocytes engraft and expand in the human subject and treat the one or more liver diseases by reducing the severity and/or frequency of symptoms, elimination of symptoms and/or underlying cause, preventing the occurrence of symptoms and/or their underlying cause, and/or improving or remediating damage caused by the disease.

All patents, patent applications and publications mentioned herein are hereby incorporated by reference for all purposes in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

### Embodiments

Accordingly, embodiments of the present subject matter described herein may be beneficial alone or in combinations, with one or more other aspects or embodiments. Without limiting the present description, certain non-limiting embodiments of the disclosure, numbered consecutively, are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below:

1. A method of producing hepatocytes, the method comprising:

administering ex vivo manipulated cells that generate hepatocytes to an animal bioreactor such that hepatocytes are expanded in the liver of the animal, optionally wherein the expanded hepatocytes comprise at least 70% of the total hepatocyte population of the animal within 8-16 weeks after administration; and

isolating the expanded hepatocytes from the animal.

2. The method of embodiment 1, wherein the ex vivo manipulation comprises culturing the hepatocyte-generating cells with at least one agent that promotes growth, regeneration, survival and/or engraftment of the hepatocytes in the animal bioreactor.

3. The method of embodiment 2, wherein the at least one or more agents comprise one or more antibodies, one or more small molecules, and/or one or more nucleic acids, optionally a c-MET and/or epidermal growth factor (EGFR) antibody.

4. The method of any of the preceding embodiments, wherein the expanded hepatocytes are human hepatocytes.

5. The method of any of the preceding embodiments, wherein the animal bioreactor comprises a genetically modified animal.

6. The method of any of the preceding embodiments, wherein the animal bioreactor is FAH-deficient.

7. The method of any of the preceding embodiments, wherein the animal bioreactor comprises a mouse, rat or pig.

8. The method of any of the preceding embodiments, wherein the ex vivo manipulated hepatocyte-generating cells are injected into the animal bioreactor.

9. The method of any of the preceding embodiments, wherein the ex vivo manipulated hepatocyte-generating cells are injected intravenously into the animal bioreactor.

10. The method of any of the preceding embodiments, wherein the ex vivo manipulated hepatocyte-generating cells are administered to an organ of the animal bioreactor, optionally via intra-splenic injection, intra-portal injection or direct injection into the liver of the animal bioreactor.

11. The method of any of the preceding embodiments, wherein greater than 10% rates of hepatocyte repopulation are achieved in the animal bioreactor.

12. The method of any of the preceding embodiments, wherein greater than 40% rates of hepatocyte repopulation are achieved in the animal bioreactor.

13. The method of any of the preceding embodiments, wherein the hepatocyte-generating cells are obtained from a commercial source or isolated from live subjects or cadavers, or primary human hepatocytes pre-expanded in vitro, and then subject to ex vivo manipulation.

14. The method of any of the preceding embodiments, wherein the ex vivo manipulation comprises culturing the hepatocyte-generating cells with the at least one agent for 1 minute to 2 days prior to administration to the animal bioreactor.

15. The method of any of the preceding embodiments, wherein the ex vivo manipulation further comprises the step of rocking the hepatocyte-generating cells incubated with the at least one agent.

16. The method of any of the preceding embodiments, further comprising the step of administering NTBC to the animal bioreactor before and/or after administration of ex vivo manipulated hepatocyte-generating cells.

17. The method of any of the preceding embodiments, wherein the ex vivo manipulated hepatocyte-generating cells are expanded in the animal bioreactor for 4 to 16 weeks, optionally 6 to 10 weeks, optionally less than 8 weeks.

18. The method of any of the preceding embodiments, wherein the expanded hepatocytes comprise at least 40% of the total hepatocyte population of the animal bioreactor.

19. The method of any of the preceding embodiments, further comprising isolating the expanded hepatocytes and subjecting the isolated expanded hepatocytes to further ex vivo manipulation, optionally wherein the ex vivo manipulation comprises culturing the isolated expanded hepatocytes with at least one agent that promotes growth, regeneration, survival and/or engraftment of hepatocytes.

20. A population of expanded hepatocytes produced by the method of any of the preceding embodiments.

21. The population of expanded hepatocytes according to embodiment 20, wherein the hepatocytes are healthier, engraft better and/or are more proliferative than hepatocytes produced from hepatocyte-generating cells not cultured with the at least one agent.

22. An animal bioreactor, or liver thereof, comprising expanded ex vivo manipulated human hepatocytes, wherein the human hepatocytes comprise more than 40% of the liver cell volume of the animal bioreactor and/or more than 40% of liver hepatocytes of the animal bioreactor.

23. A method of treating and/or preventing one or more liver diseases or disorders in a subject in need thereof, the method comprising administering to the subject expanded hepatocytes produced by the method of any of the preceding embodiments or human hepatocytes isolated from the animal bioreactor of embodiment 22.

24. The method of embodiment 23, wherein the liver disease is a chronic liver disease or acute liver disease.

25. The method of embodiments 23 or 24, wherein the liver disease is cirrhosis; acute-on-chronic liver failure (ACLF); drug- or poisoning-induced liver failure; an inborn metabolic liver disease; Crigler-Najjar syndrome type 1; familial hypercholesterolemia; Factor VII deficiency; Factor VIII deficiency (Hemophilia A); Phenylketonuria (PKU); Glycogen storage disease type I; infantile Refsum's disease; Progressive familial intrahepatic cholestasis type 2; hereditary tyrosinemia type 1; a urea cycle defect; acute liver failure; acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including alcoholic hepatitis, hepatic encephalopathy, cirrhosis; and/or acute-on-chronic liver disease caused alcohol consumption, drug ingestion, and/or hepatitis B flare ups.

26. The method of any of embodiments 23 to 25, wherein the hepatocytes are administered through portal vein infusion, umbilical vein infusion, direct splenic capsule injection, splenic artery infusion, intraperitoneal injection, lymph nodes injection, optionally wherein the hepatocytes comprise encapsulated hepatocytes.

27. The method of any of embodiments 23 to 26, further comprising the step of administering to the subject one or more agents that promote growth, regeneration, survival and/or engraftment of hepatocytes in the subject.

28. The method of embodiment 27, wherein the one or more agents comprise one or more antibodies, one or more small molecules, and/or one or more nucleic acids.

29. The method of embodiment 27 or 28, wherein the at least one agent comprises a c-MET antibody, optionally wherein the c-MET antibody is human-specific.

30. The method of any of embodiments 27 to 29, wherein the one or more agents are administered to the subject one, two or more times, optionally with and/or at different times than the hepatocytes.

31. A kit comprising hepatocyte-generating cells (e.g., human hepatocytes) and/or at least one agent that promotes growth, regeneration, survival and/or engraftment of hepatocytes, optionally comprising instructions for performing any of the preceding methods.

32. A method of producing hepatocytes, the method comprising:

manipulating hepatocyte-generating cells by contacting the cells ex vivo with at least one agent that promotes growth, regeneration, survival and/or engraftment;

transplanting the ex vivo manipulated cells into an in vivo bioreactor under conditions suitable for engraftment; and

maintaining the in vivo bioreactor under conditions suitable to expand the engrafted cells and produce hepatocytes, optionally increasing engraftment and/or repopulation efficiency by at least 10% as compared to a corresponding method lacking the ex vivo manipulation.

33. The method of embodiment 32, wherein the manipulating comprises agitating a vessel containing the hepatocyte-generating cells and the at least one agent, optionally wherein the agitating comprises rocking.

34. The method of embodiment 33, wherein the method further comprises separating the at least one agent from the ex vivo manipulated cells prior to the transplanting.

35. The method of embodiment 34, wherein the separating comprises removing the at least one agent and/or isolating the ex vivo manipulated cells, optionally wherein the separating comprises centrifugation and/or aspirating.

35. The method of any of embodiments 32 to 35, further comprising isolating the expanded hepatocytes.

36. The method of any of embodiments 32 to 25, wherein the produced hepatocytes are human hepatocytes, optionally wherein the hepatocyte-generating cells comprise primary human hepatocytes.

37. The method of any of embodiments 32 to 36, wherein the at least one agent comprises an agonist that specifically binds to a growth factor receptor.

38. The method of embodiment 37, wherein the agonist comprises a small molecule or an antibody.

39. The method of any of embodiments 37 or 38, wherein the growth factor receptor is c-MET and/or EGFR.

40. The method of any of embodiments 32 to 39, wherein the at least one agent comprises a c-MET agonist antibody and/or an EGFR agonist antibody.

41. The method of embodiments 32 to 40, wherein the engrafted cells are expanded for a period from 2 to 16 weeks.

42. The method of any embodiments 32 to 41, wherein the expanded hepatocytes comprise at least 50% of the total hepatocyte population of the in vivo bioreactor.

43. The method of any of embodiments 32 to 42, wherein the in vivo bioreactor comprises an endogenous liver injury, optionally wherein the in vivo bioreactor is genetically modified to comprise the endogenous liver injury.

44. The method of any of embodiments 32 to 43, wherein the in vivo bioreactor is immunosuppressed, optionally wherein the in vivo bioreactor is genetically modified to be immunosuppressed.

45. The method of any of embodiments 32 to 44, wherein the in vivo bioreactor is a mouse, rat or pig comprising a FAH deficiency, an IL-2Rγ deficiency, a RAG1 deficiency, a RAG2 deficiency, or any combination thereof.

46. The method of embodiment 45, wherein the in vivo bioreactor is a rodent or pig comprising a FAH, RAG1 and/or RAG2, and IL-2Rγ deficiency (FRG).

47. The method of any of embodiments 32 to 46, further comprising administering NTBC to the bioreactor before and/or after administration of ex vivo manipulated hepatocyte-generating cells.

48. The method of any of embodiments 32 to 47, wherein the ex vivo manipulated hepatocyte-generating cells are administered to an organ of the in vivo bioreactor, optionally via intra-splenic injection, intra-portal injection or direct injection into the liver of the in vivo bioreactor.

49. The method of any of embodiments 32 to 48, wherein the hepatocyte-generating cells are obtained from a commercial source or isolated from live subjects or cadavers, or primary human hepatocytes pre-expanded in vitro, and then subject to ex vivo manipulation.

50. The method of any of embodiments 32 to 49, wherein the ex vivo manipulation comprises culturing the hepatocyte-generating cells with the at least one agent for 1 minute to 2 days prior to administration to the in vivo bioreactor.

51. A method of treating a subject for a liver disease, the method comprising:

administering ex vivo manipulated cells that generate hepatocytes to the subject in an amount effective to engraft and expand in vivo thereby treating the liver disease in a subject.

52. The method of embodiment 51, further comprising contacting hepatocyte-generating cells with at least one agent that promotes growth, regeneration, survival and/or engraftment to produce the ex vivo manipulated cells.

53. The method of embodiments 51 or 52, further comprising expanding the ex vivo manipulated cells in an in vivo bioreactor prior to administration to the subject.

54. The method of any of embodiments 51 to 53, wherein the liver disease is cirrhosis; acute-on-chronic liver failure (ACLF); drug- or poisoning-induced liver failure; an inborn metabolic liver disease; Crigler-Najjar syndrome type 1; familial hypercholesterolemia; Factor VII deficiency; Factor VIII deficiency (Hemophilia A); Phenylketonuria (PKU); Glycogen storage disease type I; infantile Refsum's disease; Progressive familial intrahepatic cholestasis type 2; hereditary tyrosinemia type 1; a urea cycle defect; acute liver failure; acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including alcoholic hepatitis, hepatic encephalopathy, cirrhosis; and/or acute-on-chronic liver disease caused alcohol consumption, drug ingestion, and/or hepatitis B flare ups.

55. The method of any of embodiments 51 to 54, wherein the liver disease is an inherited disorder.

56. The method of any of embodiments 51 to 55, wherein the liver disease comprises liver failure.

57. The method of any of embodiments 51 to 56, wherein, the liver disease comprises a liver-related enzyme deficiency.

58. The method of any of embodiments 51 to 57, wherein the liver disease is hereditary tyrosinemia.

59. The method of any of embodiments 51 to 58, wherein the treatment results in at least prolonged survival of the subject, optionally as compared to survival of a comparable subject not administered the ex vivo manipulated cells.

60. Use of cells produced by any of the methods or systems of the preceding embodiments for the treatment of liver disease.

61. Use of a population of cells according to embodiments 20 or 21 in the treatment of liver disease.

62. The use of embodiments 56 or 57, wherein the liver disease is cirrhosis; acute-on-chronic liver failure (ACLF); drug- or poisoning-induced liver failure; an inborn metabolic liver disease; Crigler-Najjar syndrome type 1; familial hypercholesterolemia; Factor VII deficiency; Factor VIII deficiency (Hemophilia A); Phenylketonuria (PKU); Glycogen storage disease type I; infantile Refsum's disease; Progressive familial intrahepatic cholestasis type 2; hereditary tyrosinemia type 1; a urea cycle defect; acute liver failure; acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including alcoholic hepatitis, hepatic encephalopathy, cirrhosis; and/or acute-on-chronic liver disease caused alcohol consumption, drug ingestion, and/or hepatitis B flare ups.

What is claimed is:

1. A method of producing hepatocytes, the method comprising:
administering ex vivo manipulated cells that generate hepatocytes to an animal bioreactor such that hepatocytes are expanded in the liver of the animal, optionally wherein the expanded hepatocytes comprise at least 70% of the total hepatocyte population of the animal within 8-16 weeks after administration; and
isolating the expanded hepatocytes from the animal.

2. The method of claim 1, wherein the ex vivo manipulation comprises culturing the hepatocyte-generating cells with at least one agent that promotes growth, regeneration, survival and/or engraftment of the hepatocytes in the animal bioreactor.

3. The method of claim 2, wherein the at least one or more agents comprise one or more antibodies, one or more small molecules, and/or one or more nucleic acids.

4. The method of claim 3, wherein the at least one agent comprises a hepatocyte growth factor receptor (c-MET) and/or epidermal growth factor (EGFR).

5. The method of claim 1, wherein the expanded hepatocytes are human hepatocytes.

6. The method of claim 1, wherein the animal bioreactor comprises a genetically modified animal.

7. The method of claim 1, wherein the animal bioreactor comprises a mouse, rat or pig.

8. The method of claim 1, wherein the ex vivo manipulated hepatocyte-generating cells are administered to an organ of the animal bioreactor, optionally via intra-splenic injection, intra-portal injection or direct injection into the liver of the animal bioreactor.

9. The method of claim 1, wherein greater than 10% rates of hepatocyte repopulation are achieved in the animal bioreactor.

10. The method of claim 9, wherein greater than 40% rates of hepatocyte repopulation are achieved in the animal bioreactor.

11. The method of claim 2, wherein the ex vivo manipulation comprises culturing the hepatocyte-generating cells with the at least one agent for 1 minute to 2 days prior to administration to the animal bioreactor.

12. The method of claim 2, wherein the ex vivo manipulation further comprises the step of rocking the hepatocyte-generating cells incubated with the at least one agent.

13. The method of claim 1, wherein the ex vivo manipulated hepatocyte-generating cells are expanded in the animal bioreactor for 4 to 16 weeks, optionally 6 to 10 weeks, optionally less than 8 weeks.

14. The method of claim 1, wherein the expanded hepatocytes comprise at least 40% of the total hepatocyte population of the animal bioreactor.

15. The method of claim 2, further comprising isolating the expanded hepatocytes and subjecting the isolated expanded hepatocytes to further ex vivo manipulation, optionally wherein the ex vivo manipulation comprises culturing the isolated expanded hepatocytes with at least one agent that promotes growth, regeneration, survival and/or engraftment of hepatocytes.

16. A population of expanded hepatocytes produced by the method of claim 1.

17. The population of expanded hepatocytes according to claim 16, wherein the hepatocytes are healthier, engraft better and/or are more proliferative than hepatocytes produced from hepatocyte-generating cells not cultured with the at least one agent.

18. An animal bioreactor, or liver thereof, comprising expanded ex vivo manipulated human hepatocytes, wherein the human hepatocytes comprise more than 40% of the liver cell volume of the animal bioreactor and/or more than 40% of liver hepatocytes of the animal bioreactor.

19. A method of treating and/or preventing one or more liver diseases or disorders in a subject in need thereof, the method comprising administering to the subject expanded hepatocytes produced by the method of any of the preceding claims or human hepatocytes isolated from the animal bioreactor of claim 18.

20. The method of claim 19, wherein the liver disease is a chronic liver disease or acute liver disease.

21. The method of claim 20 wherein the liver disease is cirrhosis; acute-on-chronic liver failure (ACLF); drug- or poisoning-induced liver failure; an inborn metabolic liver disease; Crigler-Najjar syndrome type 1; familial hypercholesterolemia; Factor VII deficiency; Factor VIII deficiency (Hemophilia A); Phenylketonuria (PKU); Glycogen storage disease type I; infantile Refsum's disease; Progressive familial intrahepatic cholestasis type 2; hereditary tyrosinemia type 1; a urea cycle defect; acute liver failure; acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including alcoholic hepatitis, hepatic encephalopathy, cirrhosis; and/or acute-on-chronic liver disease caused alcohol consumption, drug ingestion, and/or hepatitis B flare ups.

22. The method of claim 19, wherein the hepatocytes are administered through portal vein infusion, umbilical vein infusion, direct splenic capsule injection, splenic artery infusion, intraperitoneal injection, lymph nodes injection, optionally wherein the hepatocytes comprise encapsulated hepatocytes.

23. A kit comprising hepatocyte-generating cells and/or at least one agent that promotes growth, regeneration, survival and/or engraftment of hepatocytes, optionally comprising instructions for generating expanded hepatocytes.

24. A method of producing hepatocytes, the method comprising:
manipulating hepatocyte-generating cells by contacting the cells ex vivo with at least one agent that promotes growth, regeneration, survival and/or engraftment;
transplanting the ex vivo manipulated cells into an in vivo bioreactor under conditions suitable for engraftment; and
maintaining the in vivo bioreactor under conditions suitable to expand the engrafted cells and produce hepatocytes, optionally increasing engraftment and/or repopulation efficiency by at least 10% as compared to a corresponding method lacking the ex vivo manipulation.

25. A method of treating a subject for a liver disease, the method comprising:
administering ex vivo manipulated cells that generate hepatocytes to the subject in an amount effective to engraft and expand in vivo thereby treating the liver disease in a subject.

26. The method of claim 25, further comprising contacting hepatocyte-generating cells with at least one agent that promotes growth, regeneration, survival and/or engraftment to produce the ex vivo manipulated cells.
